SEQUENCE LISTING (1 sequence)

SEQ ID NO: 1 - protein, 15 aa, Oryctolagus cuniculus
CKPEFNNHKDYAQEK

(19) United States
(12) Patent Application Publication
(10) Pub. No.: US 2023/0302013 A1
(43) Pub. Date: Sep. 28, 2023

(54) METHOD OF TREATING VIRAL-INDUCED COGNITIVE DYSFUNCTION BY TARGETING LEAKY RyR2 CHANNELS

(71) Applicant: The Trustees of Columbia University in the City of New York, New York, NY (US)

(72) Inventor: Andrew R. MARKS, New York, NY (US)

(21) Appl. No.: 18/166,875

(22) Filed: Feb. 9, 2023

Related U.S. Application Data
(60) Provisional application No. 63/308,198, filed on Feb. 9, 2022.

(51) Int. Cl. A61K 31/554; A61K 31/675

(52) U.S. Cl. CPC A61K 31/554; A61K 31/675

(57) ABSTRACT

A method for treating cognitive dysfunction by administering a therapeutically effective amount of a calcium channel stabilizer to a subject in need thereof. The preferred calcium channel stabilizers include a 1,4-benzothiazepine moiety, including those of the general structural formula:

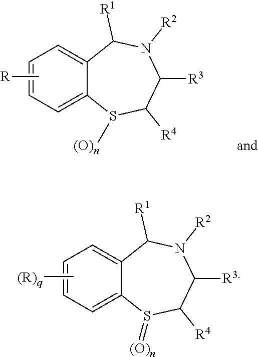

The method is useful with various types of respiratory viruses, including for coronaviruses, COVID, SARS and MERS. The method of treatment increases RyR2-Castabin2 binding in cardiac muscle of the subject while also decreasing open probability (Po) of RyR2 protein in the subject. All of these effects act to at least partially offset cognitive dysfunctions such as a deficit in attention, executive functioning, language, processing speed, memory, and combinations thereof.

### CROSS-REFERENCE TO RELATED APPLICATION

This application claims priority to U.S. Provisional Application No. 63/308,198 filed on Feb. 9, 2022, the entire content of which is incorporated herein by reference thereto.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 1,815 byte XML file named “44010-115US-PAT-CU22201.xml” created on Feb. 9, 2023.

### TECHNICAL FIELD

The present subject matter relates to a method for treating cognitive dysfunction associated with viral infection, e.g., SARS-CoV-19, by targeting leaky RyR2 channels.

### BACKGROUND

Patients suffering from COVID-19 exhibit multi-organ system failure involving not only pulmonary but also cardiovascular, neural and other systems. The pleiotropy and complexity of the organ system failures both complicate the care of COVID-19 patients, and contribute to a great extent to the morbidity and mortality of the pandemic. Clinical data indicate that severe COVID-19 most commonly manifests as viral pneumonia-induced acute respiratory distress syndrome (ARDS). Respiratory failure results from severe inflammation in the lungs, which arises when COVID-19 infects lung cells and damages them. Cardiac manifestations are multifactorial, and include hypoxia, hypotension, enhanced inflammatory status, angiotensin-converting enzyme 2 (ACE2) receptor downregulation, endogenous catecholamine adrenergic status, and direct viral myocardial damage. Moreover, patients with underlying cardiovascular disease or comorbidities, including congestive heart failure, hypertension, diabetes, and pulmonary diseases, are more susceptible to infection by SARS CoV-2, with higher mortality. In addition to respiratory and cardiac manifestation, it has been reported that of patients with COVID-19 develop neurological symptoms, including headache, disturbed consciousness, and paresthesia. Brain tissue edema, stroke, partial neuronal degeneration in deceased patients, and neuronal encephalitis have also been reported. Furthermore, another pair of frequent symptoms of infection by SARS-CoV-2 are hyposmia and hypogeusia, the loss of the ability to smell and taste, respectively.

There is a need for treatments of people who have been infected with COVID-19 or similar viruses to reduce or limit brain damage along with the neurological manifestations that result from such damage.

### SUMMARY OF THE INVENTION

The present disclosure provides a method for treating cognitive dysfunction associated with a respiratory virus infection. This method comprises administering a therapeutically effective amount of a Ryanodine Receptor (RyR) calcium channel stabilizer to a subject in need thereof. An exemplary RyR calcium channel stabilizer comprises a 1,4-benzothiazepine moiety.

The method described herein is useful for treating cognitive dysfunction associated with various types of respiratory viruses, including and specifically for coronaviruses. In particular, the respiratory virus may be selected from acute respiratory syndrome (SARS) coronavirus (SARS-CoV), SARS-CoV-2 (COVID-19), and Middle East Respiratory Syndrome (MERS) virus, respiratory syncytial virus (RSV), influenza virus, parainfluenza virus (NV), pneumovirus (PMV), metapneumovirus (MPV), respirovirus, and rubulavirus.

In some embodiments, the present method of treatment can decrease calcium leak from a RyR2 channel of the subject. Such treatment can also increase RyR2-Castabin2 binding in the subject. The treatment can also decrease open probability (Po) of the RyR2 channel protein in the subject. All of these effects act to at least partially offset cognitive dysfunctions such as a deficit in attention, executive functioning, language, processing speed, memory, and combinations thereof in a COVID-19 patient.

In some embodiments, a compound that can be used in the present methods include those of the general structural formula:

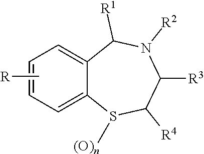

wherein,
   n is 0, 1, or 2;
   R is located at one or more positions on the benzene ring; each R is independently selected from the group consisting of H, halogen, —OH, —NH₂, —NO₂, —CN, —N₃, —SO₃H, acyl, alkyl, alkoxyl, alkylamino, cycloalkyl, heterocyclyl, heterocyclylalkyl, alkenyl, (hetero-)aryl, (hetero-)arylthio, and (hetero-)arylamino; wherein each acyl, alkyl, alkoxyl, alkylamino, cycloalkyl, heterocyclyl, heterocyclylalkyl, alkenyl, (hetero-)aryl, (hetero-)arylthio, and (hetero-)arylamino may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
   R¹ is selected from the group consisting of H, oxo, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
   R² is selected from the group consisting of —C═O(R⁵), —C═S(R⁶), —SO₂R⁷, —POR⁸R⁹, —(CH₂)ₘ—R¹⁰, alkyl, aryl, heteroaryl, cycloalkyl, cycloalkylalkyl, and heterocyclyl; wherein each alkyl, aryl, heteroaryl, cycloalkyl, cycloalkylalkyl, and heterocyclyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
   R³ is selected from the group consisting of H, —CO₂Y, —CONY, acyl, alkyl, alkenyl, aryl, cycloalkyl, and heterocyclyl; wherein each acyl, alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl; and wherein Y is selected from the group consisting of H, alkyl, aryl, cycloalkyl, and heterocyclyl;
   R⁴ is selected from the group consisting of H, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
   R⁵ is selected from the group consisting of —NR¹⁶, NHNHR¹⁶, NHOH, —OR¹⁵, —CONH₂NHR¹⁶, —CO₂R¹⁵, CONR¹⁶, —CH₂X, acyl, alkenyl, aryl, cycloalkyl, cycloalkylalkyl, heterocyclyl, and heterocyclylalkyl; wherein each acyl, alkenyl, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
   R⁶ is selected from the group consisting of —OR¹⁵, —NHNR¹⁶, —NHOH, —NR¹⁶, —CH₂X, acyl, alkenyl, alkyl, aryl, cycloalkyl, cycloalkylalkyl, heterocyclyl, and heterocyclylalkyl; wherein each acyl, alkenyl, alkyl, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
   R⁷ is selected from the group consisting of —OR¹⁵, —NR¹⁶, —NHNHR¹⁶, —NHOH, —CH₂X, alkyl, aryl, cycloalkyl, cycloalkylalkyl, heterocyclyl, and heterocyclylalkyl; wherein each alkyl, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
   R⁸ and R⁹ independently are selected from the group consisting of OH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
   R¹⁰ is selected from the group consisting of NH₂, —OH, —SO₂R¹¹, —NHSO₂R¹¹, —C═O(R¹²), —NHC═O(R¹²), —OC═O(R¹²), and —POR¹³R¹⁴;
   —R¹¹, R¹², R¹³, and R¹⁴ independently are selected from the group consisting of H, —OH, —NH₂, —NHNH₂, —NHOH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
   X is selected from the group consisting of halogen, —CN, —CO₂R¹⁵, —CONR¹⁶, —NR¹⁶, —OR¹⁵, —SO₂R⁷, and —POR⁸R⁹; and
   R¹⁵ and R¹⁶ independently are selected from the group consisting of H, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
or a pharmaceutically-acceptable salt, hydrate, solvate, complex, or prodrug thereof.

In some embodiments, compound that can be used in the present methods include those of the general structural formula:

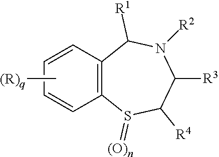

wherein:
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
   R¹ is alkyl, alkenyl, aryl, alkylaryl, cycloalkyl, heteroaryl, or heterocyclyl, each of which is independently substituted or unsubstituted; or H;
   R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
   R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
   Y is alkyl, aryl, alkylaryl, cycloalkyl, heteroaryl, or heterocyclyl, each of which is independently substituted or unsubstituted; or H;
   R⁴ is alkyl, alkenyl, aryl, alkylaryl, cycloalkyl, heteroaryl, or heterocyclyl, each of which is independently substituted or unsubstituted; or H;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
   each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
   each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
   each R¹⁰ is —NR¹⁵R¹⁶, —OH, —SO₂R¹¹, —NHSO₂R¹¹, —C(═O)(R¹²), NHC═O(R¹²), —OC═O(R¹²), or —P(═O)R¹³R¹⁴;
   each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
   each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
   each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
n is 0, 1, or 2;
q is 0, 1, 2, 3, or 4;
t is 1, 2, 3, 4, 5, or 6; and
m is 1, 2, 3, or 4,
   or a pharmaceutically-acceptable salt, hydrate, solvate, complex, or prodrug thereof.

In some embodiments, the compound is represented by the structure

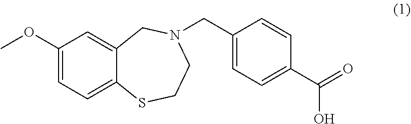 [SMILES: COc1ccc2c(c1)CN(Cc1ccc(C(=O)O)cc1)CCS2]

or a pharmaceutically-acceptable salt thereof.

The calcium channel stabilizer can be administered to the subject, for example in a pharmaceutical composition that further comprises at least one pharmaceutically acceptable excipient.

### BRIEF DESCRIPTION OF DRAWINGS

Various embodiments will now be described in detail with reference to the accompanying drawings.

FIGS. 1A-1D show increased oxidative stress, inflammatory and adrenergic signaling in brains of COVID-19 patients. FIG. 1A is a bar graph depicting the GSSG/GSH ratio and kynurenic acid (KYNA) ELISA signal from control (n=6) and COVID-19 (n=6) tissue lysates. Ctx—Cortex. CB—cerebellum. Data are mean±SD. *p<0.05 control vs COVID-19. FIG. 1B are western blots showing phospho-SMAD3 and total SMAD3 from control (n=4) and COVID-19 (n=7) brain lysates. FIG. 1C is a bar graph depicting quantification of pSMAD3/SMAD3 from Western blot signals of FIG. 1B. FIG. 1D shows CAMKII and PKA activity of brain tissue lysates. Data are mean±SD. *p<0.05 control vs COVID-19.

FIGS. 2A-2D show hyperphosphorylation of Tau but normal APP processing in COVID-19 brains. FIG. 2A are immunoblots of brain (Ctx, CB) lysates that were separated by 4-20% PAGE. Immunoblots were developed for pAMPK, AMPK, GSK3β, pGSK3β (T216), APP, BACE1, and GAPDH loading control. The numbers (1-10) above immunoblots refer to patient numbers listed in Table 1. FIG. 2B are bar graphs showing quantification of pAMPK, pGSK3β, APP/GAPDH, and BACE1/GAPDH from Western blots of FIG. 2A. Data are mean±SD. *p<0.05 control vs COVID-19; **p<0.05 CB vs. Ctx; #p<0.05 COVID (Young) vs. COVID (Old). FIG. 2C are immunoblots of brain lysates showing total Tau and Tau phosphorylation on residues S199, S202/T205, S214, S262, and S356. FIG. 2D is a bar graph showing quantification phosphorylated Tau at the residues shown on Western blots of FIG. 2C. Data are mean±SD. *p<0.05 control vs COVID-19; **p<0.05 CB vs. Ctx; #p<0.05 COVID (Young) vs. COVID (Old).

FIGS. 3A-3E show Dysregulation of calcium-handling proteins in COVID-19 brains. FIG. 3A are Western blots depicting RyR2 oxidation, PKA phosphorylation, and calstabin2 or NOX2 bound to the channel from brain (Ctx, CB) lysates. FIG. 3B are bar graphs quantifying DNP/RyR2, pS2808/RyR2, and calstabin2 and NOX2 bound to the channel from the Western blots. Data are mean±SD. *p<0.05 control vs COVID-19; #p<0.05 COVID-19 vs COVID-19+Compound 1. FIG. 3C are bar graphs showing ³[H]ryanodine binding from immunoprecipitated RyR2 at 150 nM Ca²⁺ as a percent of maximum binding (Ca²⁺=20 Data are mean±SD. *p<0.05 control vs COVID-19; #p<0.05 COVID-19 vs COVID-19+Compound 1. FIG. 3D are western blots showing the levels of GCPII, Calbindin, and GAPDH loading control in brain (Ctx, CB). FIG. 3E are bar graphs quantifying GCPII/GAPDH and Calbindin/GAPDH from the Western blots. Data are mean±SD. *p<0.05 control vs COVID-19.

FIG. 4 is a sketch illustrating how SARS-CoV-2 infection results in leaky RyR2 that is likely to contribute to cardiac, pulmonary, and cognitive dysfunction.

FIGS. 5A-5D show a biochemical signature of leaky RyR2 channels in COVID-19 heart and lung tissues. FIG. 5A are western blots showing activation of TGFβ signaling in COVID-19 heart and lung lysates as indicated by increased phospho-SMAD3/total SMAD3 from control and COVID-19 heart and lung lysates. FIG. 5B are western blots showing increased NOX2 bound to RyR2 in both the heart and lung lysates from COVID-19 patients.

FIGS. 5C and 5D are western blots depicting RyR2 oxidation, PKA phosphorylation, and calstabin2 bound to the channel from heart (FIG. 5C) and lung (FIG. 5D).

### DETAILED DESCRIPTION OF THE INVENTION

### Definitions

The following definitions are provided for the purpose of understanding the present subject matter and for constructing the appended patent claims.

It is noted that, as used in this specification and the appended claims, the singular forms “a”, “an”, and “the” include plural references unless the context clearly dictates otherwise.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

As used herein, the term “salt” has the same meaning as commonly understood to one of ordinary skill in the art. Specifically, a salt is a chemical compound consisting of an ionic assembly of positively charged cations and negatively charged anions.

As used herein, the term “hydrate” has the same meaning as commonly understood to one of ordinary skill in the art. Specifically, a hydrate is a compound with extra water molecules that are part of its structure.

As used herein, the term “solvate” has the same meaning as commonly understood to one of ordinary skill in the art. Specifically, a solvate is a compound formed by the interaction of a solvent and a solute.

As used herein, the term “complex” has the same meaning as commonly understood to one of ordinary skill in the art. Specifically, a complex is a molecular entity formed by loose association involving two or more component molecular entities (ionic or uncharged), or the corresponding chemical species. The bonding between the components is normally weaker than in a covalent bond.

As used herein, the term “prodrug” has the same meaning as commonly understood to one of ordinary skill in the art. Specifically, a prodrug is a precursor of a drug—a compound that, on administration to a subject, undergoes metabolic processes that convert the compound to the drug.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

### Ryanodine Receptors: Excitation-Contraction Coupling (ECC) Process

The sarcoplasmic reticulum (SR) is a structure in cells that functions, among other things, as a specialized intracellular calcium (Ca²⁺) store. Ryanodine receptors (RyRs) are channels in the SR, which open and close to regulate the release of Ca²⁺ from the SR into the intracellular cytoplasm of the cell. Release of Ca²⁺ into the cytoplasm from the SR increases cytoplasmic Ca²⁺ concentration. Open probability of RyRs refers to the likelihood that a RyR is open at any given moment, and therefore capable of releasing Ca²⁺ into the cytoplasm from the SR.

The RyR is the major Ca²⁺ release channel on the SR responsible for excitation-contraction coupling (ECC) in striated muscle. Among the three known RyR isoforms (RyR1, RyR2 and RyR3), RyR1 is widely expressed and is the predominant isoform expressed in mammalian skeletal muscle. RyR2 is also widely expressed and is the predominant form found in cardiac muscle. RyR3 expression is low in adult skeletal muscle. RyR subtypes exhibit a high degree of structural and functional homology. The subtypes form a large sarcoplasmic membrane complex, consisting of four monomers that constitute a Ca²⁺ release channel associated with proteins, such as kinases, phosphatases, phosphodiesterases, and other regulatory subunits.

Ca²⁺ release from the SR is modulated by several RyR binding proteins. Calmodulin, a key mediator of Ca²⁺ signaling, exerts both positive and negative effects on RyR open probability. Calstabin1 (FKBP12) and calstabin2 (FKBP12.6) stabilize the closed state of RyR1 and RyR2, respectively. Calstabin1 associates predominantly with skeletal muscle RyR1, while cardiac muscle RyR2 has the highest affinity for Calstabin2.

Mutations in RYR1 or RYR2 can cause decreased binding of Calstabin1 and Calstabin2, respectively. Stress-induced post-translational modifications of RyRs including PKA phosphorylation, oxidation, and nitrosylation also can cause decreased binding of Calstabins to RyR channels. Genetic mutations and/or stress-induced posttranslational modifications of the channel can result in dissociation of Calstabin, leading to leaky channels that exhibit a pathologic increase in the open probability under resting conditions. The SR Ca²⁺ leak leads to a reduction in SR Ca²⁺ content, with less Ca²⁺ available for release and consequently weaker muscle contractions. The intracellular calcium leak has distinct pathological consequences depending on which tissue is involved.

RyR calcium channel modulators, for example, Rycal® compounds are small molecules that can, for example, bind to leaky RyR subunits, restore Calstabin binding, and repair the channel leak. In some embodiments, Rycal® compounds bind to leaky RyR channels, restore Calstabin binding, and fix the channel leak without blocking the RyR channel. In some embodiments, Rycal® compounds are capable of fixing a leak in RyR channels, for example, RyR2 channels. In some embodiments, the compositions described herein enhance association and/or inhibit dissociation of RyR and Calstabin (e.g., RyR2 and Calstabin2).

### Therapeutic Methods

Provided herein are methods for treating cognitive dysfunction associated with a respiratory virus infection. The methods comprise administering a therapeutically effective amount of a compound, e.g., a calcium channel stabilizer as described herein to a subject in need thereof. The subjects to be treated are those that are known to have or have had a respiratory viral infection.

In some embodiments, provided herein method of treating cognitive dysfunction associated with a respiratory virus infection, the method comprising administering a therapeutically effective amount of a calcium channel stabilizer to a subject in need thereof, the calcium channel stabilizer comprising a 1,4-benzothiazepine moiety.

In some embodiments, the respiratory virus is a coronavirus. In some embodiments, the respiratory virus is a severe acute respiratory syndrome (SARS) coronavirus. In some embodiments, the respiratory virus is a severe acute respiratory syndrome (SARS) coronavirus (SARS-CoV). In some embodiments, the respiratory virus is severe acute respiratory syndrome (SARS)-CoV-2 (COVID-19). In some embodiments, the respiratory virus is a Middle East Respiratory Syndrome (MERS) virus. In some embodiments, the respiratory virus is a respiratory syncytial virus (RSV). In some embodiments, the respiratory virus is influenza virus. In some embodiments, the respiratory virus is parainfluenza (a virus (NV). In some embodiments, the respiratory virus is pneumovirus (PMV). In some embodiments, the respiratory virus is metapneumovirus (MPV). In some embodiments, the respiratory virus is a respirovirus. In some embodiments, the respiratory virus is a rubulavirus.

The calcium channel stabilizer can be administered at any time once it is known that the subject has COVID-19, or any time after the subject begins experiencing neurological dysfunction associated with COVID-19. It is possible to administer the calcium channel stabilizer weeks, months or years after the subject recovers from COVID, although it is believed that the earlier the administration occurs, the sooner the subject's leaky RyR Ca′ channels can be repaired, thus offsetting or preventing cognitive dysfunction which can otherwise lead to long-term neurologic consequences.

In some embodiments, the respiratory virus is SARS-CoV-2. SARS-CoV-2 infection can be associated with adrenergic and oxidative stress and activation of the TGF-β signaling pathway in the brains of patients who have succumbed to COVID-19. One consequence of this hyper-adrenergic and oxidative state can be the development of Tau pathology normally associated with Alzheimer's disease. In some embodiments, Tau pathology can be linked to Ca²⁺ dysregulation associated with leaky RyR channels in the brain.

It has now been found that SARS-CoV-2 infection activates inflammatory signaling and oxidative stress pathways resulting in hyperphosphorylation of Tau, but normal amyloid precursor protein processing in COVID-19 patient cortex and cerebellum. In some embodiments, reduced calbindin expression was found in both cortex and rendering both tissues vulnerable to Ca²⁺-mediated pathology. Moreover, COVID-19 cortex and cerebellum exhibited RyR Ca²⁺ release channels with the biochemical signature of “leaky” channels and increased open probability, consistent with pathological intracellular Ca²⁺ leak. RyR2 channels were oxidized, associated with increased NADPH oxidase 2 (NOX2), and were PKA hyperphosphorylated on Serine 2808, both of which cause loss of the stabilizing subunit Calstabin2 from the channel complex promoting leaky RyR2 channels in COVID-19 patient brains. Furthermore, ex vivo treatment of COVID-19 patient brain samples with Compound 1, a Rycal® compound which binds to leaky RyR channels, fixed the channel leak.

TGF-β belongs to a family of cytokines involved in the formation of cellular fibrosis by promoting epithelial-to-mesenchymal transition, fibroblast proliferation, and differentiation. TGF-β activation has been shown to induce fibrosis in the lungs and other organs by activation of the SMAD-dependent pathway. TGF-β/SMAD3 activation can lead to NOX2/4 translocation to the cytosol and its association with RyR channels, promoting oxidization of the channels and depletion of the stabilizing subunit Calstabin in skeletal muscle and in heart Alteration of Ca²⁺ signaling can be important in COVID-19-infected patients with cardiovascular/neurological diseases due, in part, to the multifactorial RyR2 remodeling following the cytokine storm, increased TGF-β activation, and increased oxidative stress. As demonstrated herein, SARS-CoV-2-infected patients exhibited a hyperadrenergic state, which leads to hyper-phosphorylation of RyR2 channels, promoting pathological remodeling of the channel and exacerbating defective Ca²⁺ regulation.

Both the cortex and cerebellum of SARS-CoV-2-infected patients exhibited a reduced expression of the Ca²⁺ buffering protein, calbindin. Decreased calbindin is expected to render these tissues more vulnerable to the cytosolic Ca²⁺ overload. This finding is in accordance with studies showing reduced calbindin expression levels in Purkinje cells and the CA2 hippocampal region of Alzheimer's Disease patients and in cortical pyramidal cells of aged individuals with Tau pathology. In contrast to the findings in the brains of COVID-19 patients in the present disclosure, calbindin was not reduced in the cerebellum of Alzheimer's Disease patients, possibly protecting these cells from Alzheimer's Disease pathology.

Leaky RyR channels, leading to increased mitochondrial Ca²⁺ overload and ROS production and oxidative stress, have been shown to contribute to the development of Tau pathology associated with Alzheimer's disease. Studies of the effects of COVID-19 on the central nervous system have found memory deficits and biological markers similar to those seen in Alzheimer's Disease patients. Data is presented to demonstrate increased activity of enzymes responsible for phosphorylating Tau (pAMPK, pGSK3β), as well as increased phosphorylation at multiple sites on Tau in COVID-19 patient brains. The Tau phosphorylation observed in these samples exhibited some differences from what is typically observed in Alzheimer's Disease, occurring in younger patients and in areas of the brain, specifically the cerebellum, that usually do not demonstrate Tau pathology in Alzheimer's Disease patients. Taken together, these data suggest a potential contributing mechanism to the development of Tau pathology in COVID-19 patients involving oxidative overload driven RyR2 channel dysfunction. Furthermore, it is anticipated that these pathological changes could be a significant contributing factor to the neurological manifestations of COVID-19 and in particular “the “brain fog” associated with long COVID and represent a potential therapeutic target for ameliorating these symptoms. For example, Tau pathology in the cerebellum could explain why many hospitalized COVID-19 patients experienced coordination deficits. The data presented herein also raises the possibility that prior COVID-19 infection could be a potential risk factor for developing Alzheimer's Disease in the future.

Taken together, the results show that SARS-CoV-2 infection can activate biochemical pathways that are linked to the Tau pathology associated with Alzheimer's Disease, and that implicate leaky RyR Ca²⁺ channels. Targeting leaky RyRs can be a potential therapeutic target for the neurological complications associated with COVID-19.

### Compounds

The compounds of use in the methods of the present disclosure include any one of a wide variety of calcium channel stabilizers that comprise a 1,4-benzothiazepine moiety.

Calcium channel stabilizer can include compounds commonly referred to as Rycal® compounds, such as 1,4-benzothiazepines and related structures, described in U.S. Pat. No. 8,710,045, issued on Apr. 29, 2014, and U.S. Pat. No. 8,022,058, issued on Sep. 20, 2011, the contents of each of which are incorporated by reference herein.

In an embodiment, a calcium channel stabilizer comprises the following structural formula:

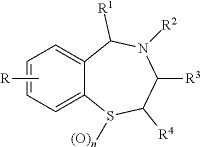

wherein,
   n is 0, 1, or 2;
   R is located at one or more positions on the benzene ring; each R is independently selected from the group consisting of H, halogen, —OH, —NH₂, —NO₂, —CN, —N₃, —SO₃H, acyl, alkyl, alkoxyl, alkylamino, cycloalkyl, heterocyclyl, heterocyclylalkyl, alkenyl, (hetero-)aryl, (hetero-)arylthio, and (hetero-)arylamino; wherein each acyl, alkyl, alkoxyl, alkylamino, cycloalkyl, heterocyclyl, heterocyclylalkyl, alkenyl, (hetero-)aryl, (hetero-)arylthio, and (hetero-)arylamino may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
   R¹ is selected from the group consisting of H, oxo, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
   R² is selected from the group consisting of —C═O(R⁵), —C═S(R⁶), —SO₂R⁷, —POR⁸R⁹, —(CH₂)ₘ—R¹⁰, alkyl, aryl, heteroaryl, cycloalkyl, cycloalkylalkyl, and heterocyclyl; wherein each alkyl, aryl, heteroaryl, cycloalkyl, cycloalkylalkyl, and heterocyclyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
   R³ is selected from the group consisting of H, —CO₂Y, —CONY, acyl, alkyl, alkenyl, aryl, cycloalkyl, and heterocyclyl; wherein each acyl, alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl; and wherein Y is selected from the group consisting of H, alkyl, aryl, cycloalkyl, and heterocyclyl;
   R⁴ is selected from the group consisting of H, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
   R⁵ is selected from the group consisting of —NR¹⁶, NHNHR¹⁶, NHOH, —CONH₂NHR¹⁶, —CO₂R¹⁵, —CONR¹⁶, —CH₂X, acyl, alkenyl, aryl, cycloalkyl, cycloalkylalkyl, heterocyclyl, and heterocyclylalkyl; wherein each acyl, alkenyl, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
   R⁶ is selected from the group consisting of —OR¹⁵, —NHNR¹⁶, —NHOH, —NR¹⁶, —CH₂X, acyl, alkenyl, alkyl, aryl, cycloalkyl, cycloalkylalkyl, heterocyclyl, and heterocyclylalkyl; wherein each acyl, alkenyl, alkyl, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
   R⁷ is selected from the group consisting of —OR¹⁵, —NR¹⁶, —NHNHR¹⁶, —NHOH, —CH₂X, alkyl, aryl, cycloalkyl, cycloalkylalkyl, heterocyclyl, and heterocyclylalkyl; wherein each alkyl, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
   R⁸ and R⁹ independently are selected from the group consisting of OH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
   R¹⁰ is selected from the group consisting of NH₂, —OH, —SO₂R¹¹, —NHSO₂R¹¹, —C═O(R¹²), —NHC═O(R¹²), —OC═O(R¹²), and —POR¹³R¹⁴;
   R¹¹, R¹², R¹³, and R¹⁴ independently are selected from the group consisting of H, —OH, —NH₂, —NHNH₂, —NHOH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
   X is selected from the group consisting of halogen, —CN, —CO₂R¹⁵, —CONR¹⁶, —NR¹⁶, —OR¹⁵, —SO₂R⁷, and —POR⁸R⁹; and
   R¹⁵ and R¹⁶ independently are selected from the group consisting of H, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
or a pharmaceutically-acceptable salt, hydrate, solvate, complex, or prodrug thereof.

In an embodiment, a calcium channel stabilizer comprises the following structural formula I:

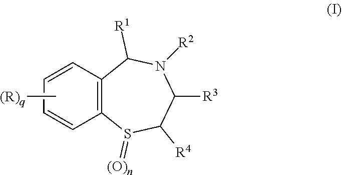

wherein,
n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, —C(═O)(R¹²), —NHC═O(R¹²), —OC═O(R¹²), or —P(═O)R¹³R¹⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹; and
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
t is 1, 2, 3, 4, 5, or 6;
m is 1, 2, 3, or 4;
   or a pharmaceutically-acceptable salt thereof.

In some embodiments, the present disclosure provides compounds of Formula I-a:

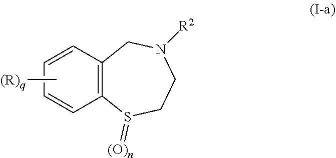

wherein:
n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)R¹², NH(C═O)R¹², —O(C═O)R¹², or —P(═O)R¹³R¹⁴;
M is 0, 1, 2, 3, or 4;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is halogen, —CN, —CO₂R¹⁵, —C(═O)NR¹⁵R¹⁶, —NR¹⁵R¹⁶, —OR¹⁵, —SO₂R⁷, or —P(═O)R⁸R⁹; and
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
or a pharmaceutically-acceptable salt thereof.

In some embodiments, the present disclosure provides a compound of formula I-a, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1, or 2.

In some embodiments, the present disclosure provides a compound of formula I-a, wherein R₂ is —C═O(R⁵), —C═S(R⁶), —SO₂R⁷, —P(═O)R⁸R⁹, or —(CH₂)ₘ—R¹⁰.

In some embodiments, the present disclosure provides a compound of formula I-b:

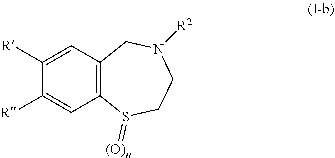

wherein
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰; and
n is 0, 1, or 2;
or a pharmaceutically-acceptable salt thereof.

In some embodiments, the present disclosure provides a compound of formula I-b, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-b, wherein R² is —C═O(R⁵), —C═S(R⁶), —SO₂R⁷, —P(═O)R⁸R⁹, or —(CH₂)ₘ—R¹⁰.

In some embodiments, the present disclosure provides a compound formula of

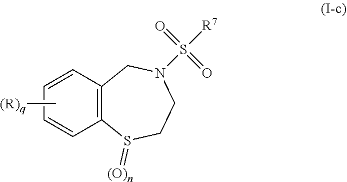

n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X; or a pharmaceutically-acceptable salt thereof.

In some embodiments, the present disclosure provides a compound of formula I-c, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —OS(═O)₂CF₃, Ph, —NHCH₂Ph, —C(═O)Me, —OC(═O)Me, morpholinyl, or propenyl; and n is 0, 1, or 2.

In some embodiments, the present disclosure provides a compound of formula I-c, wherein R⁷ is alkyl, alkenyl, aryl, cycloalkyl, cycloalkylalkyl, heterocyclyl, or heterocyclylalkyl, each of which is independently substituted or unsubstituted; or —OH or —NR¹⁵R¹⁶.

In some embodiments, the present disclosure provides a compound of formula of I-d:

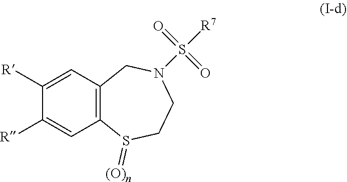

n is 0, 1, or 2;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X,
   or a pharmaceutically-acceptable salt thereof.

In some embodiments, the present disclosure provides a compound of formula wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-d, wherein R⁷ is alkyl, alkenyl, aryl, cycloalkyl, cycloalkylalkyl, heterocyclyl, or heterocyclylalkyl, each of which is independently substituted or unsubstituted; or —OH, or —NR¹⁵R¹⁶.

In some embodiments, the present disclosure provides a compound of formula of I-e:

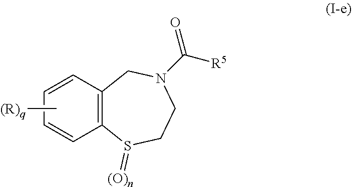

n is 0, 1, or 2;
q is 0, 1, 2, 3, or 4;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃; and
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group, or a pharmaceutically-acceptable salt thereof.

In some embodiments, the present disclosure provides a compound of formula I-e, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1, or 2.

In some embodiments, the present disclosure provides a compound of formula I-e, wherein R₅ is alkyl, alkenyl, aryl, cycloalkyl, cycloalkylalkyl, heterocyclyl, or heterocyclylalkyl, each of which is independently substituted or unsubstituted; or —NR¹⁵R¹⁶, NHOH, —OR¹⁵, or —CH₂X.

In some embodiments, the present disclosure provides a compound of formula of I-f:

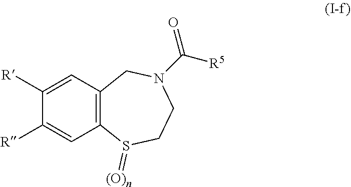

n is 0, 1, or 2;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —C(═O)NHNR¹⁵R¹⁶, —CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group,
or a pharmaceutically-acceptable salt thereof.

In some embodiments, the present disclosure provides a compound of formula I-f, wherein R′ and R″ are each independently H, halogen, —OH, OMe, —NH₂, —NO₂, —CN, —CF₃, —OCF₃, —N₃, —S(═O)₂C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-f, wherein R⁵ is alkyl, alkenyl, aryl, cycloalkyl, cycloalkylalkyl, heterocyclyl, or heterocyclylalkyl, each of which is independently substituted or unsubstituted; or —NR¹⁵R¹⁶, —NHOH, —OR¹⁵, or —CH₂X.

In some embodiments, the present disclosure provides a compound of formula of I-g:

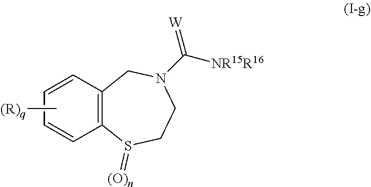

wherein
n is 0, 1, or 2;
q is 0, 1, 2, 3, or 4;
W is S or O;
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded may form a heterocycle that is substituted or unsubstituted,
   or a pharmaceutically-acceptable salt thereof.

In some embodiments, the present disclosure provides a compound of formula I-g, wherein each R is independently selected from the group consisting of H, halogen, —OH, OMe, —NH₂, —NO₂, —CN, —CF₃, —OCF₃, —N₃, —S(═O)₂C₁-C₄alkyl, —S(═O)C₁-C₄alkyl, —OS(═O)₂CF₃, Ph, —NHCH₂Ph, —C(═O)Me, —OC(═O)Me, morpholinyl and propenyl; and n is 0, 1, or 2.

In some embodiments, the present disclosure provides a compound of formula I-g, wherein R¹⁵ and R¹⁶ are each independently alkyl, alkylamino, aryl, cycloalkyl, cycloalkylalkyl, heterocyclyl, and heterocyclylalkyl, each of which is independently substituted or unsubstituted; or H, OH, or NH₂; or R¹⁵ and R¹⁶ together with the N to which they are bonded form a heterocycle that is substituted or unsubstituted.

In some embodiments, the present disclosure provides a compound of formula I-g, wherein W is O or S.

In some embodiments, the present disclosure provides a compound of formula of I-h:

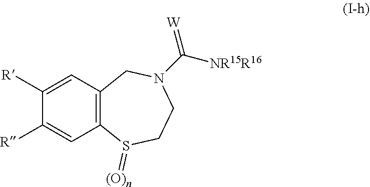

n is 0, 1, or 2;
W is S or O;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃,
or a pharmaceutically-acceptable salt thereof.

In some embodiments, the present disclosure provides a compound of formula wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. Preferably, R′ is H or OMe, and R″ is H.

In some embodiments, the present disclosure provides a compound of formula I-h, wherein R¹⁵ and R¹⁶ are each independently alkyl, alkylamino, aryl, cycloalkyl, cycloalkylalkyl, heterocyclyl, or heterocyclylalkyl, each of which is independently substituted or unsubstituted; or H, OH, NH₂; or R¹⁵ and R¹⁶ together with the N to which R¹⁵ and R¹⁶ are bonded form a heterocycle that is substituted or unsubstituted.

In some embodiments, the present disclosure provides a compound of formula I-g, wherein W is O or S.

In some embodiments, the present disclosure provides a compound of formula of I-i:

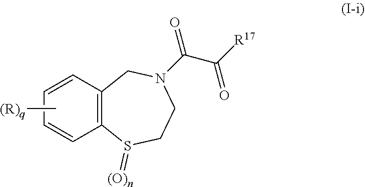

wherein
R¹⁷ is alkenyl, aryl, cycloalkyl, cycloalkylalkyl, heterocyclyl, and heterocyclylalkyl, each of which is independently substituted or unsubstituted; or —NR¹⁵R¹⁶, —NHNR¹⁵R¹⁶, —NHOH, —OR¹⁵, or —CH₂X;
n is 0, 1, or 2;
q is 0, 1, 2, 3, or 4; and
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃,
or a pharmaceutically-acceptable salt thereof.

In some embodiments, the present disclosure provides a compound of formula I-i, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1, or 2.

In some embodiments, the present disclosure provides a compound of formula I-i, wherein R¹⁷ is —NR¹⁵R¹⁶ or —OR¹⁵. In some embodiments, R¹⁷ is —OH, —OMe, —Net, —NHEt, —NHPh, —NH₂, or —NHCH₂pyridyl.

In some embodiments, the present disclosure provides a compound of formula of I-j:

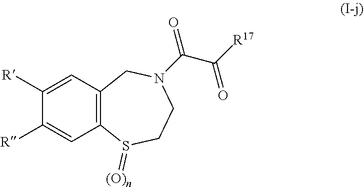

R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
R¹⁷ is selected from the group consisting of —NR¹⁵R¹⁶, —NHOH, —OR¹⁵, —CH₂X, alkenyl, aryl, cycloalkyl, cycloalkylalkyl, heterocyclyl, and heterocyclylalkyl; wherein each alkenyl, aryl, cycloalkyl, cycloalkylalkyl, heterocyclyl, and heterocyclylalkyl may be substituted or unsubstituted;
n is 0, 1, or 2,
   or a pharmaceutically-acceptable salt thereof.

In some embodiments, the present disclosure provides a compound of formula I-j, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-j, wherein R¹⁷ is —NR¹⁵R¹⁶ or —OR¹⁵. In some embodiments, R¹⁷ is —OH, —OMe, —Net, —NHEt, —NHPh, —NH₂, or —NHCH₂pyridyl.

In some embodiments, the present disclosure provides a compound of formula I-k or I-k-1:

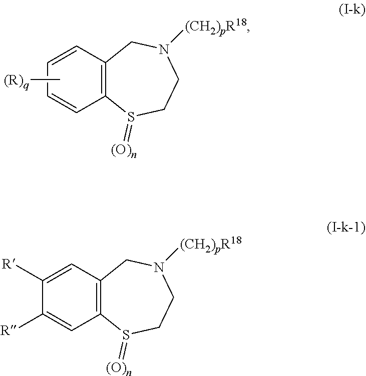

wherein
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
R¹⁸ is alkyl, aryl, cycloalkyl, or heterocyclyl, each of which is independently substituted or unsubstituted; or —NR¹⁵R¹⁶, —C(═O)NR¹⁵R¹⁶, —(C═O)OR¹⁵, or —OR¹⁵;
q is 0, 1, 2, 3, or 4;
p is 1, 2, 3, 4, 5, 6, 7, 8 9, or 10; and
n is 0, 1, or 2,
or a pharmaceutically-acceptable salt thereof.

In some embodiments, the present disclosure provides a compound of formula I-k, wherein each R is independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R is OMe at position 7 of the benzothiazepine ring.

In some embodiments, the present disclosure provides a compound of formula I-k-1, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-k or I-k-1, wherein R¹⁸ is —NR¹⁵R¹⁶, —(C═O)OR¹⁵, —OR¹⁵, alkyl that is substituted or unsubstituted, or aryl that is substituted or unsubstituted. In some embodiments, m is 1, and R¹⁸ is Ph, C(═O)OMe, C(═O)OH, aminoalkyl, NH₂, NHOH, or NHCbz. In other embodiments, m is 0, and R¹⁸ is C₁-C₄ alkyl. In other embodiments, R¹⁸ is Me, Et, propyl, and butyl. In some embodiments, m is 2, and R¹⁸ is pyrrolidine, piperidine, piperazine, or morpholine. In some embodiments, m is 3, 4, 5, 5, 7, or 8, and R¹⁸ is a fluorescent labeling group selected from bodipy, dansyl, fluorescein, rhodamine, Texas red, cyanine dyes, pyrene, coumarins, Cascade Blue™, Pacific Blue, Marina Blue, Oregon Green, 4′,6-Diamidino-2-phenylindole (DAPI), indopyra dyes, lucifer yellow, propidium iodide, porphyrins, arginine, and variants and derivatives thereof.

In some embodiments, the present disclosure provides a compound of formula of I-1 or I-1-1:

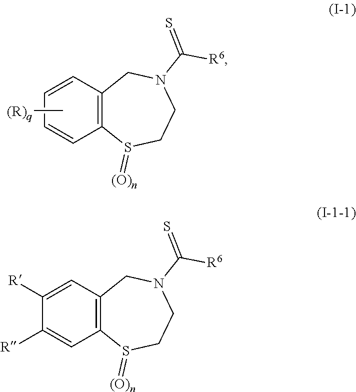

wherein
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃;
R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
q is 0, 1, 2, 3, or 4; and
n is 0, 1, or 2,
   or a pharmaceutically acceptable salt thereof.

In some embodiments, the present disclosure provides a compound of formula I-1, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R is OMe at position 7 of the benzothiazepine ring.

In some embodiments, the present disclosure provides a compound of formula I-1-1, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-1 or I-1-1, wherein R⁶ is acyl, alkenyl, alkyl, aryl, cycloalkyl, cycloalkylalkyl, heterocyclyl, and heterocyclylalkyl, each of which is independently substituted or unsubstituted; or —NR¹⁵R¹⁶, —NHNR¹⁵R¹⁶, —OR¹⁵, —NHOH, or —CH₂X. In some embodiments, R⁶ is —NR¹⁵R¹⁶. In some embodiments, R⁶ is —NHPh, pyrrolidine, piperidine, piperazine, morpholine. In some embodiments, R⁶ is alkoxyl. In some embodiments, R⁶ is —O-tBu.

In some embodiments, the present disclosure provides a compound of formula I-m or I-m-1:

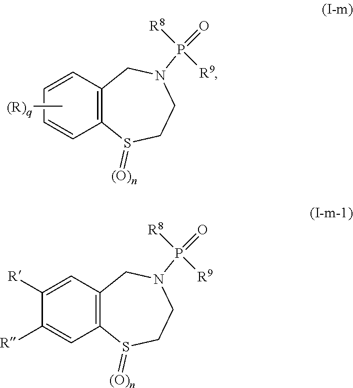

wherein
n is 0, 1, or 2;
q is 0, 1, 2, 3, or 4;
R′ and R″ are each independently acyl, alkyl, alkoxyl, alkylamino, alkylthio, cycloalkyl, aryl, heterocyclyl, heterocyclylalkyl, alkenyl, alkynyl, aryl, heteroaryl, arylthiol, heteroarylthio, arylamino, or heteroarylamino, each of which is independently substituted or substituted; or halogen, H, —OH, —NH₂, —NO₂, —CN, —CF₃, —OCF₃, —N₃, —SO₃H, —S(═O)₂alkyl, —S(═O)alkyl, or —OS(═O)₂CF₃; and
R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH,
   or a pharmaceutically-acceptable salt thereof.

In some embodiments, the present disclosure provides a compound of formula I-m, wherein each R is independently halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R is OMe at position 7 of the benzothiazepine ring.

In some embodiments, the present disclosure provides a compound of formula I-m-1, wherein R′ and R″ are each independently H, halogen, —OH, OMe, —NH₂, —NO₂, —CN, —CF₃, —OCF₃, —N₃, —S(═O)₂C₁-C₄alkyl, —S(═O)C₁-C₄alkyl, —S—C₁-C₄alkyl, —OS(═O)₂CF₃, Ph, —NHCH₂Ph, —C(═O)Me, —OC(═O)Me, morpholinyl, or propenyl; and n is 0, 1 or 2. In some embodiments, R′ is H or OMe, and R″ is H.

In some embodiments, the present disclosure provides a compound of formula I-m or I-m-1, wherein R⁸ and R⁹ are each independently alkyl, aryl, —OH, alkoxyl, or alkylamino. In some embodiments, R⁸ is C₁-C₄alkyl. In some embodiments, R⁸ is Me, Et, propyl or butyl. In some embodiments, R⁹ is aryl. In some embodiments, R⁹ is phenyl. In some embodiments, the present disclosure provides a compound of formula I-n,

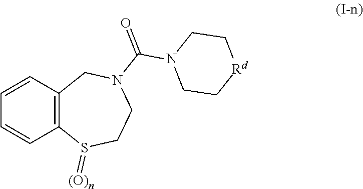

wherein:
R^{d} is CH₂, or NR^{a}; and
R^{a} is H, alkoxy, —(C₁-C₆ alkyl)-aryl, wherein the aryl is a disubstituted phenyl or a benzo[1,3]dioxo-5-yl group, or a Boc group.
   or a pharmaceutically-acceptable salt thereof.

In some embodiments, R^{a} is H.

In some embodiments, the present disclosure provides a compound of Formula I-o:

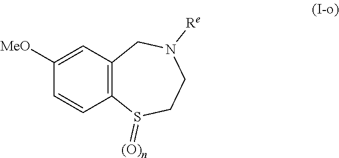

wherein:
R^{e} is —(C₁-C₆ alkyl)-phenyl, —(C₁-C₆ alkyl)-C(O)R^{b}, or substituted or unsubstituted —C₁-C₆ alkyl; and
R^{b} is —OH or —O—(C₁-C₆ alkyl),
   wherein the phenyl or the substituted alkyl is substituted with one or more of halogen, hydroxyl, —C₁-C₆ alkyl, —O—(C₁-C₆ alkyl), —NH₂, —NH(C₁-C₆ alkyl), —N(C₁-C₆ alkyl)₂, cyano, or dioxolane, or a pharmaceutically-acceptable salt thereof.

In some embodiments, the present disclosure provides a compound of Formula I-p:

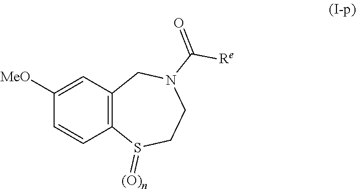

wherein R^{c} is —(C₁-C₆ alkyl)-NH₂, —(C₁-C₆ alkyl)-OR^{f}, wherein R^{f} is H or —C(O)—(C₁-C₆)alkyl, or —(C₁-C₆ alkyl)-NHR^{g}, wherein R^{g} is carboxybenzyl.

In some embodiments, the present disclosure provides compounds of Formula II or Formula III:

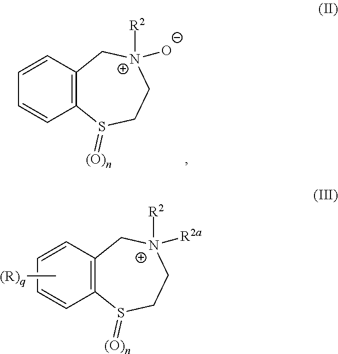

wherein:
   n is 0, 1, or 2;
   q is 0, 1, 2, 3, or 4;
   each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
   each R₂ and Rea is independently alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
   each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —NHNR¹⁵R¹⁶, —NHOH, —OR¹⁵, —C(═O)NHNR¹⁵R¹⁶, CO₂R¹⁵, —C(═O)NR¹⁵R¹⁶, —CH₂X, or alkyl substituted by at least one labeling group, selected from a fluorescent group, a bioluminescent group, a chemiluminescent group, a colorimetric group, and a radioactive labeling group;
   each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
   each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
   each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
   each R¹⁰ is —NR¹⁵R¹⁶, OH, —SO₂R¹¹, —NHSO₂R¹¹, C(═O)R¹², NH(C═O)R¹², —O(C═O)R¹², or —P(═O)R¹³R¹⁴; m is 0, 1, 2, 3, or 4;
   each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
   each X is halogen, —CN, —CO₂R¹⁵, —C(═O)NR¹⁵R¹⁶, —NR¹⁵R¹⁶, —OR¹⁵, —SO₂R⁷, or —P(═O)R⁸R⁹; and
   each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
or a pharmaceutically acceptable salt thereof.

In an embodiment, the calcium channel stabilizer is selected from the group consisting of:

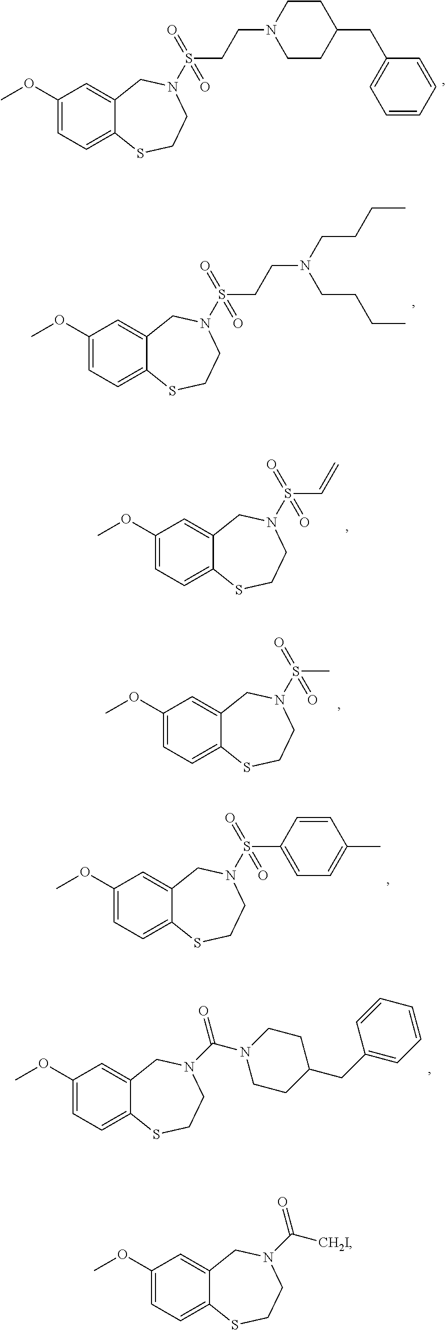

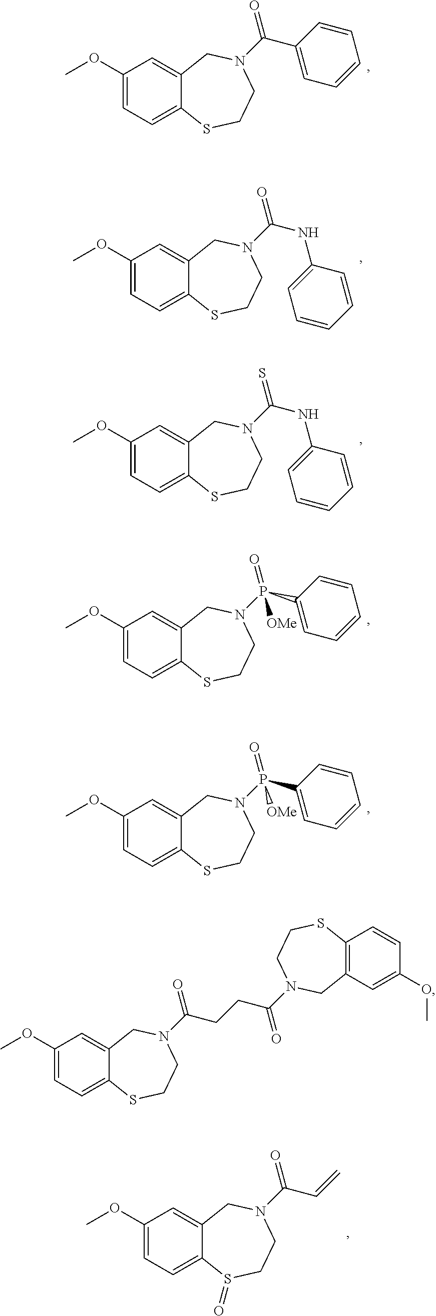

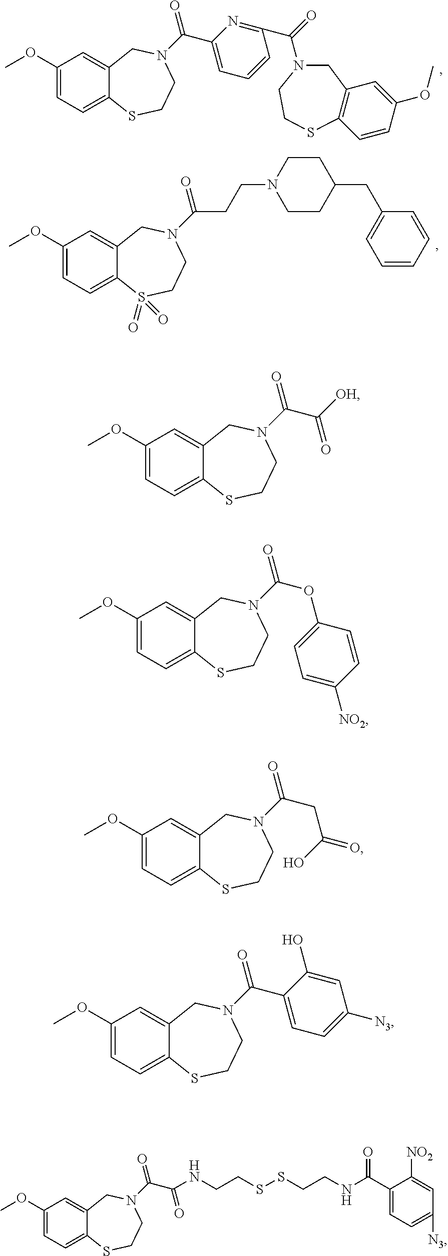

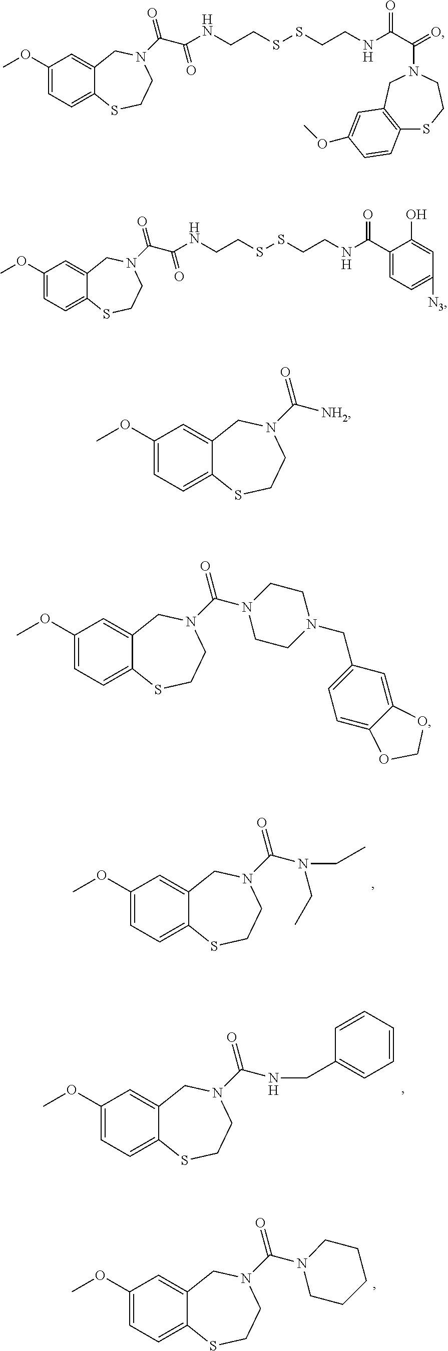

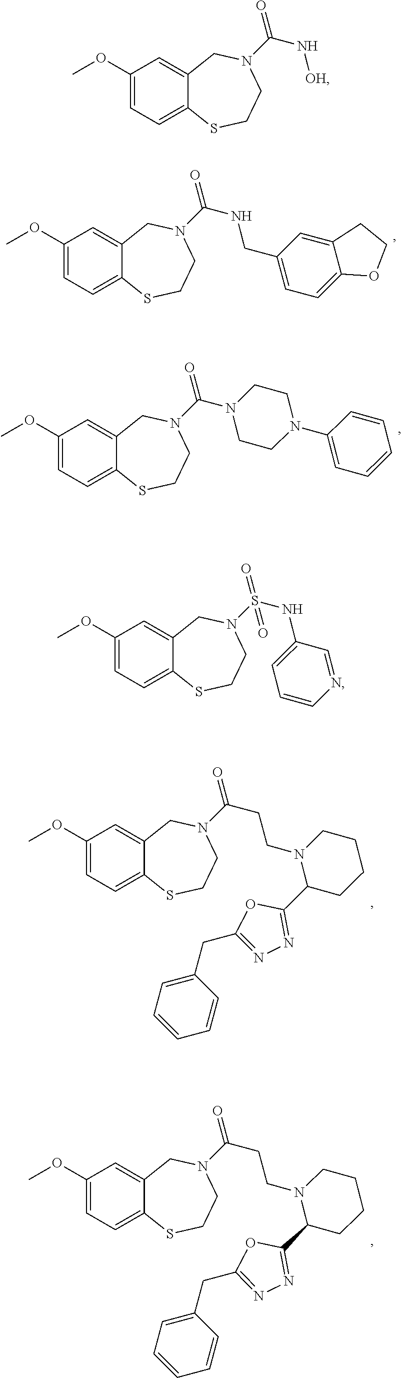 [SMILES: COc1ccc2c(c1)CN(C(=O)NO)CCS2 ; COc1ccc2c(c1)CN(C(=O)NCc1ccc3c(c1)CCO3)CCS2 ; COc1ccc2c(c1)CN(C(=O)N1CCN(c3ccccc3)CC1)CCS2 ; COc1ccc2c(c1)CN(S(=O)(=O)Nc1cccnc1)CCS2 ; COc1ccc2c(c1)CN(C(=O)CCN1CCCCC1c1nnc(Cc3ccccc3)o1)CCS2 ; COc1ccc2c(c1)CN(C(=O)CCN1CCCC[C@H]1c1nnc(Cc3ccccc3)o1)CCS2]

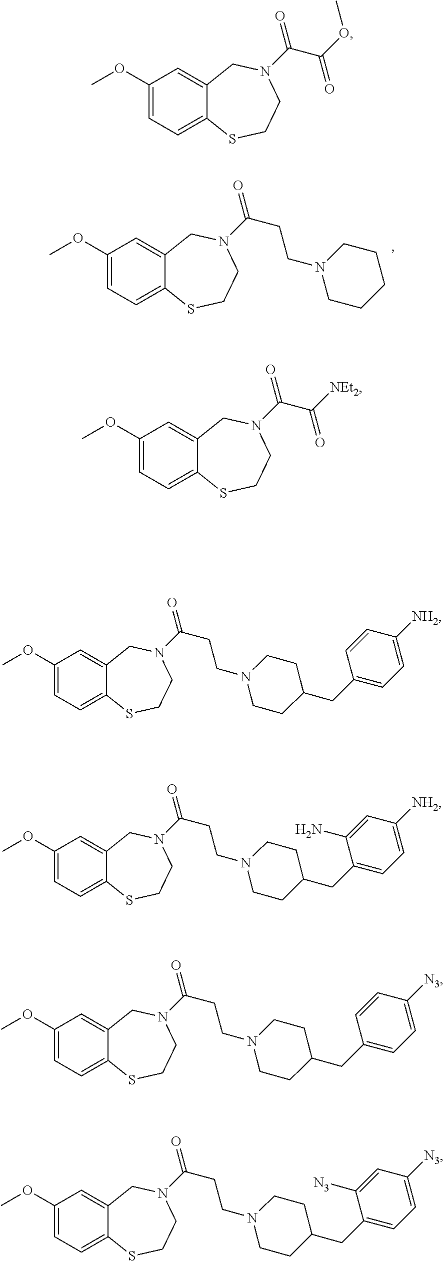

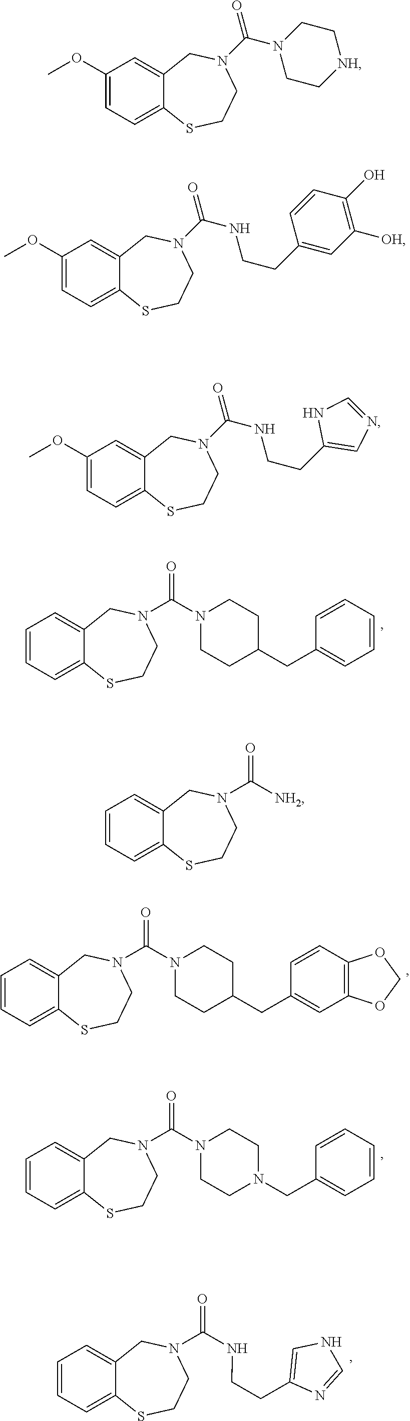

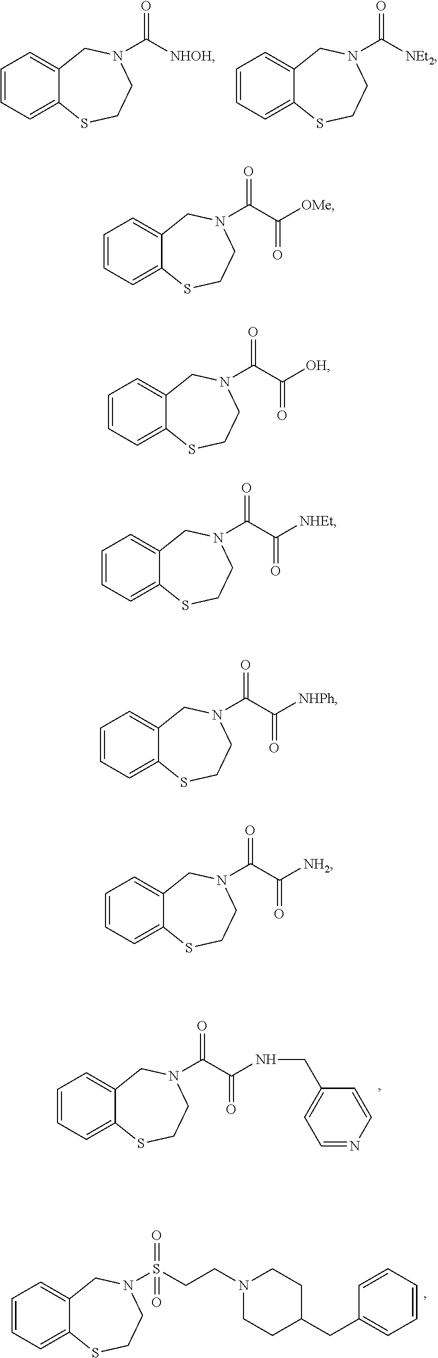

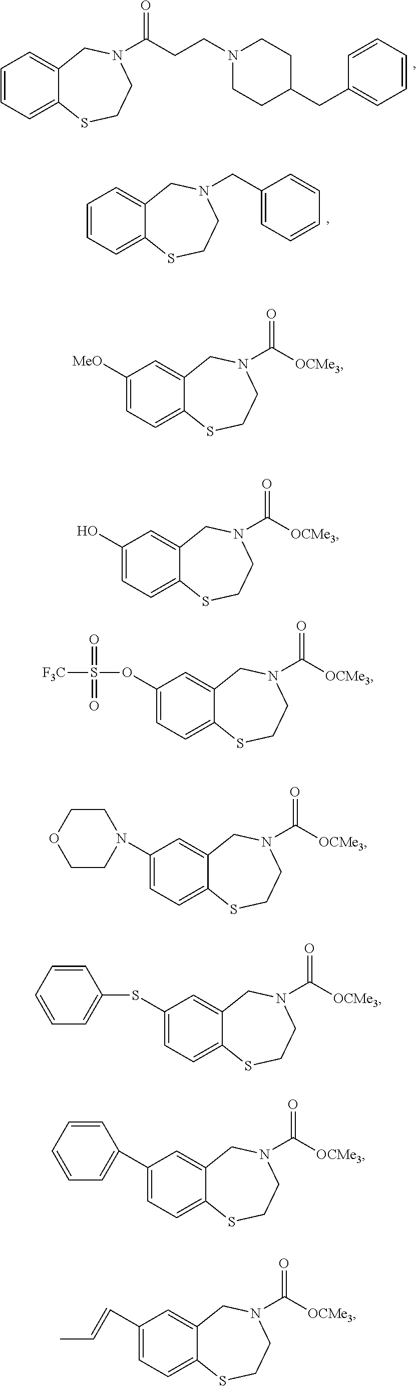

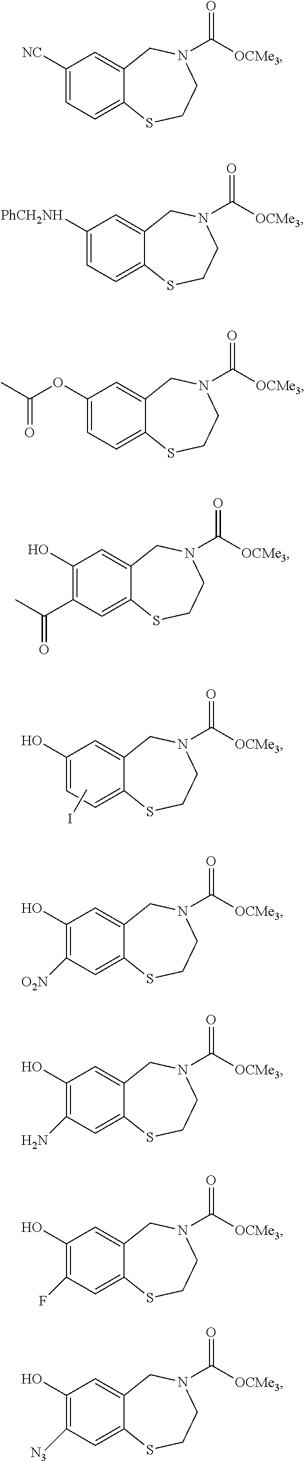

or a pharmaceutically-acceptable salt thereof.

In an embodiment, the calcium channel stabilizer is selected from the group consisting of:

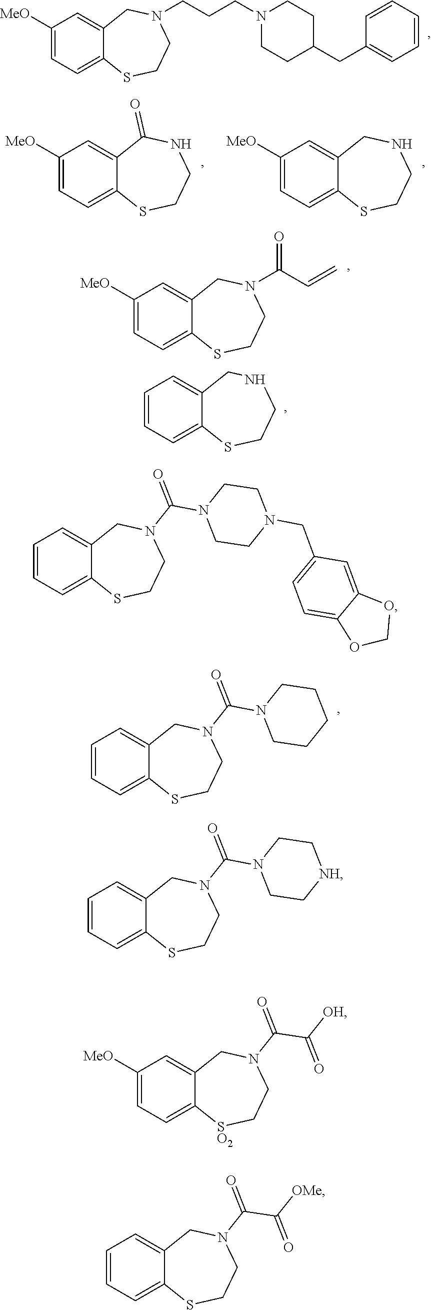

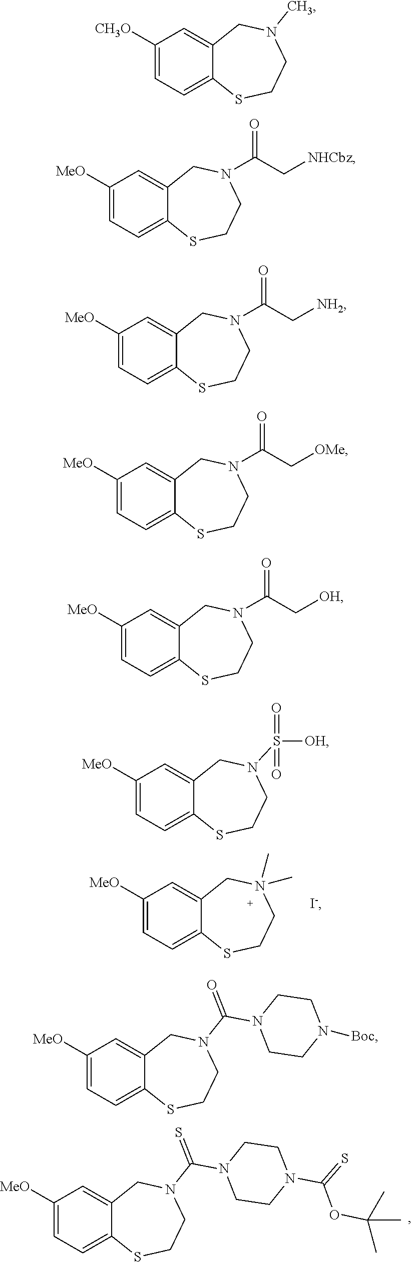

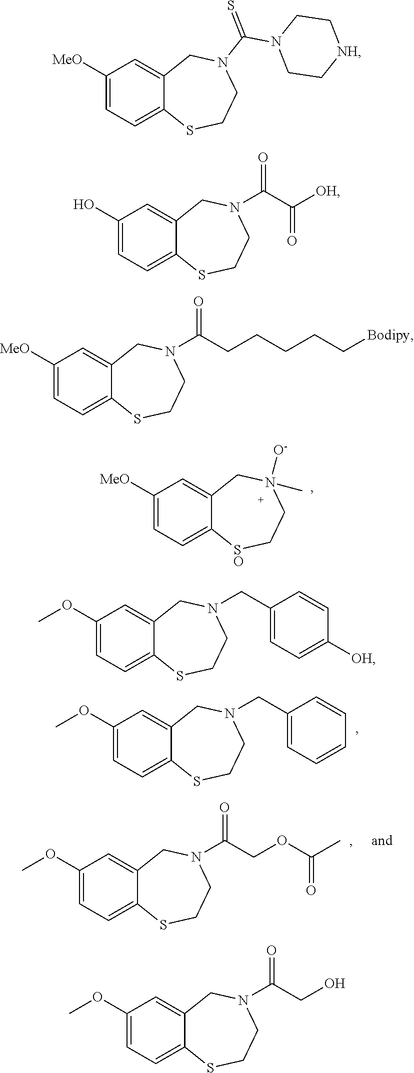

or a salt, hydrate, solvate, complex, or prodrug thereof.

In an embodiment, the calcium channel stabilizer is represented by the structure

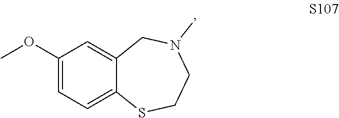

or a salt thereof, such as the HCl salt.

In an embodiment, the calcium channel stabilizer is represented by the structure

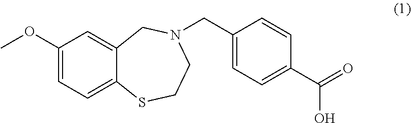 [SMILES: COc1ccc2c(c1)CN(Cc1ccc(C(=O)O)cc1)CCS2]

or a pharmaceutically-acceptable salt thereof.

### Pharmaceutical Compositions

Compounds can be formulated into pharmaceutical compositions for administration to human subjects in a biologically compatible form suitable for administration in vivo. According to another aspect, compounds are formulated into pharmaceutical compositions in admixture with a pharmaceutically acceptable diluent and/or carrier. The pharmaceutically-acceptable carrier must be “acceptable” in the sense of being compatible with the other ingredients of the composition and not deleterious to the recipient thereof. The pharmaceutically-acceptable carrier employed herein is selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. If necessary, pharmaceutical additives, such as antioxidants, aromatics, colorants, flavor-improving agents, preservatives, and sweeteners, are also added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others.

The pharmaceutical formulations can be brought into association with a carrier and/or diluent, as a suspension or solution. Optionally, one or more accessory ingredients (e.g., buffers, flavoring agents, surface active agents, and the like) also are added. The choice of carrier is determined by the solubility and chemical nature of the compounds, chosen route of administration and standard pharmaceutical practice.

In some embodiments, compounds are administered to a human or animal subject by known procedures including, without limitation, oral administration, sublingual or buccal administration, parenteral administration, transdermal administration, via inhalation or intranasally, vaginally, rectally, and intramuscularly. The compounds of the invention are administered parenterally, by epifascial, intracapsular, intracranial, intracutaneous, intrathecal, intramuscular, intraorbital, intraperitoneal, intraspinal, intrasternal, intravascular, intravenous, parenchymatous, subcutaneous or sublingual injection, or by way of catheter. In one embodiment, the agent is administered to the subject by way of delivery to the subject's muscles including, but not limited to, the subject's cardiac muscles. In an embodiment, the agent is administered to the subject by way of targeted delivery to cardiac muscle cells via a catheter inserted into the subject's heart.

For oral administration, a formulation of the compounds described herein may be presented as capsules, tablets, powders, granules, or as a suspension or solution. The formulation has conventional additives, such as lactose, mannitol, cornstarch or potato starch. The formulation also is presented with binders, such as crystalline cellulose, cellulose derivatives, acacia, cornstarch or gelatins. Additionally, the formulation is presented with disintegrators, such as cornstarch, potato starch or sodium carboxymethylcellulose. The formulation also is presented with dibasic calcium phosphate anhydrous or sodium starch glycolate. Finally, the formulation is presented with lubricants, such as talc or magnesium stearate.

For parenteral administration (i.e., administration by injection through a route other than the alimentary canal), the compounds can be combined with a sterile aqueous solution that is isotonic with the blood of the subject. Such a formulation is prepared by dissolving a solid active ingredient in water containing physiologically-compatible substances, such as sodium chloride, glycine and the like, and having a buffered pH compatible with physiological conditions, so as to produce an aqueous solution, then rendering said solution sterile. The formulation is presented in unit or multi-dose containers, such as sealed ampoules or vials. The formulation is delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, subcutaneous, or sublingual or by way of catheter into the subject's heart.

For transdermal administration, the compounds can be combined with skin penetration enhancers, such as propylene glycol, polyethylene glycol, isopropanol, ethanol, oleic acid, N-methylpyrrolidone and the like, which increase the permeability of the skin to the compounds of the invention and permit the compounds to penetrate through the skin and into the bloodstream. The compound/enhancer compositions also may be further combined with a polymeric substance, such as ethylcellulose, hydroxypropyl cellulose, ethylene/vinylacetate, polyvinyl pyrrolidone, and the like, to provide the composition in gel form, which are dissolved in a solvent, such as methylene chloride, evaporated to the desired viscosity and then applied to backing material to provide a patch.

In some embodiments, in order to prepare the pharmaceutical composition, the calcium channel stabilizer, as the active ingredient, is intimately admixed with a pharmaceutically acceptable carrier according to conventional pharmaceutical compounding techniques. Carriers are inert pharmaceutical excipients, including, but not limited to, binders, suspending agents, lubricants, flavorings, sweeteners, preservatives, dyes, and coatings. In preparing compositions in oral dosage form, any of the pharmaceutical carriers known in the art may be employed. For example, for liquid oral preparations, suitable carriers and additives include water, glycols, oils, alcohols, flavoring agents, preservatives, coloring agents, and the like. Further, for solid oral preparations, suitable carriers and additives include starches, sugars, diluents, granulating agents, lubricants, binders, disintegrating agents, and the like.

The present compositions can be provided in unit dosage forms such as tablets, pills, capsules, powders, granules, ointments, sterile parenteral solutions or suspensions, metered aerosol or liquid sprays, drops, ampules, auto-injector devices or suppositories, for oral parenteral, intranasal, sublingual or rectal administration, or for administration by inhalation or insufflation. The composition can be presented in a form suitable for daily, weekly, or monthly administration. The pharmaceutical compositions herein will contain, per dosage unit, e.g., tablet, capsule, powder, injection, teaspoonful, suppository and the like, an amount of the active ingredient necessary to deliver an effective dose. Concentrations of the calcium channel stabilizer will typically be in the range of about 1 to about 10 mg/kg and preferably about 2 to about 5 mg/kg based on the weight of the subject. A therapeutically effective amount of the calcium channel stabilizer or an amount effective to treat cognitive dysfunction associated with a respiratory virus infection may be specifically determined initially from the Example described herein and adjusted as necessary using routine methods.

### EXAMPLE

### Summary

Brain tissues from control and COVID-19 patients were analyzed for oxidative stress and inflammatory signaling pathway markers and Alzheimer's disease (AD)-linked signaling. Since defective calcium signaling and leaky ryanodine receptor/calcium (Ca′) release channels (RyR) on the endoplasmic reticuli (ER) have been linked to Alzheimer's Disease, post-translational modifications, and function of neuronal RyRs were also measured. Tau phosphorylation and activation of pathways associated with Alzheimer's Disease were detected in COVID-19 patient brains. SARS-CoV-2 infection was associated with inflammatory signaling, increased TGF-β activity, and oxidative overload in the brain. In addition, COVID-19 brain tissues exhibited increased protein kinase A (PKA) and Ca²⁺/calmodulin kinase II (CAMKII) activity. RyR2 channels in COVID-19 brains had the biochemical signature of leaky channels, and increased activity consistent with leak which can promote cognitive and behavioral defects. The RyR2 channel leak was corrected by ex vivo treatment with Rycal® Compound 1 which stabilizes the closed state of the channel. Taken together these data suggest that severe COVID-19 may be associated with Alzheimer's Disease-like neuropathology that could be the cause of brain “fog” observed in some “long-COVID” patients. Leaky RyR2 also appears to play a role in this COVID-19 neuropathology and is a therapeutic target for amelioration of some cognitive defects associated with SARS-CoV-2 infection.

### Consolidated Results and Study Design

Increased markers of oxidative stress (GSSH/GSH) were found in cortex (mesial temporal lobe) and cerebellum (cerebellar cortex, lateral hemisphere) of COVID-19 tissue. Kynurenic acid, a marker of inflammation, was increased in COVID-19 cortex and cerebellum brain lysates compared to controls which is in accordance with recent studies showing a positive correlation between kynurenic acid and cytokines and chemokines levels in COVID-19 patients.

In order to determine if SARS-CoV-2 infection also increases tissue TGF-β activity, SMAD3 phosphorylation, a downstream signal of TGFβ, was measured in control and COVID-19 tissue lysates. Phosphorylated SMAD3 (pSMAD3) levels were increased in COVID-19 cortex and cerebellum brain lysates compared to controls, indicating that SARS-CoV-2 infection increased TGF-β signaling in these tissues. Brain tissues from COVID-19 patients exhibited activation of the TGF-β pathway, despite the absence of the detectable (by immunohistochemistry and PCR) virus in these tissues. These results suggest that the TGF-β pathway is activated systemically by SARS-CoV-2, resulting in its upregulation in the brain, as well as other organs. In addition to oxidative stress, COVID-19 brain tissues also demonstrated increased PKA and CaMKII activity, most likely associated with increased adrenergic stimulation. Both PKA and CaMKII phosphorylation of Tau have been reported in tauopathies. The hallmarks of Alzheimer's Disease brain neuropathology are the formation of amyloid-β (Aβ) plaques from abnormal amyloid precursor protein (APP) processing by BACE1, as well as Tau “tangles” caused by Tau hyperphosphorylation (ADD REFS). Brain lysates from COVID-19 patients' autopsies demonstrated normal BACE1 and APP levels compared to controls. The patients analyzed in the present study were grouped by age (Young ≤58 years old, Old ≥66 years old) to account for normal, age dependent changes in APP and Tau pathology. Abnormal APP processing was only observed in brain lysates from patients with diagnosed AD. However, AMPK and GSK3β phosphorylation were increased in both the cortex and cerebellum in COVID-19 brains. Activation of these kinases in SARS-CoV-2 infected brains leads to a hyperphosphorylation of Tau consistent with Alzheimer's Disease Tau pathology in the cortex. COVID-19 brain lysates from older patients showed increased Tau phosphorylation at S199, S202, S214, S262, and S356. Lysates from younger COVID-19 patients showed increased Tau phosphorylation at S214, S262, and S356, but not at S199 and S202, demonstrating increased Tau phosphorylation in both young and old samples and suggesting a Tau pathology similar to Alzheimer's Disease in COVID-19 affected patients. Both young and old patient brains demonstrated increased Tau phosphorylation in the cerebellum, which is not typical of Alzheimer's Disease.

RyR channels can be oxidized due to the activation of the TGF-β signaling pathway. NOX2 binding to RyR2 causes oxidation of the channel which activates the channel, manifested as an increased open probability which can be assayed using ³[H]ryanodine binding. When the oxidization of the channel is at pathological levels, there is destabilization of the closed state of the channel, resulting in spontaneous Ca²⁺ release or leak. To determine the effect of the increased TGF-β signaling associated with SARS-Cov-2 infection on NOX2/RyR2 interaction, RyR2 and NOX2 were co-immunoprecipitated from brain lysates of COVID-19 patients and controls. NOX2 associated with RyR2 in brain tissues from SARS-CoV-2 infected individuals was increased compared to controls.

Given the increased oxidative stress and increased NOX2 binding to RyR2 seen in COVID-19 brains, RyR2 post-translational modifications were also investigated. Immunoprecipitated RyR2 from brain lysates demonstrated increased oxidation, PKA phosphorylation on Serine 2808, and depletion of the stabilizing protein subunit calstabin2 in SARS-CoV-2 infected tissues compared to controls. This biochemical remodeling of the channel is known as the “biochemical signature” of leaky RyR2 that is associated with destabilization of the closed state of the channel. This leads to SR/ER Ca²⁺ leak which contributes to the pathophysiology of a number of diseases including Alzheimer's Disease. RyR channel activity was determined by binding of 3[H]ryanodine, which binds only to the open state of the channel. RyR2 was immunoprecipitated from tissue lysates and ryanodine binding was measured at both 150 nM and 20 μM free Ca²⁺. RyR2 channels from SARS-CoV-2 infected brain tissue demonstrated abnormally high activity (i.e., increased ryanodine binding) compared to channels from control tissues at physiologically resting conditions (150 nM free Ca²⁺), when channels should be closed.

Cortex and cerebellum of SARS-CoV-2-infected patients also exhibited a reduced expression of the Ca²⁺ binding protein calbindin. Calbindin is typically not reduced in the cerebellum of Alzheimer's Disease patients, possibly providing some protection against Alzheimer's Disease pathology. The low levels of calbindin in the cerebellum of COVID-19 brains could therefore contribute to the observed Tau pathology in the cerebellum. An additional atypical finding in the COVID-19 brains studied in this investigation is an increased level of GCPII. This could contribute to the observed RyR PKA phosphorylation by increasing cAMP and inhibiting the metabotropic glutamate receptor type 3.

### Methods

### Human Samples

De-identified human heart, lung, and brain tissue were obtained from the COVID BioBank at Columbia University. The Columbia University BioBank functions under standard operating procedures, quality assurance, and quality control for sample collection and maintenance. Age- and sex-matched controls exhibited absence of neurological disorders and cardiovascular or pulmonary diseases. Sex, age, and pathology of patients are listed in Table 1.

TABLE 1
Sex, age, and pathology of COVID-19 patients.

| Patient | | | |
|---|---|---|---|
| Number | Sex | Age | Pathology |
| 1 | Male | 57 | Acute hypoxic-ischemic injury in the hippocampus, pons, and |
| | | | cerebellum. |
| 2 | Female | 38 | Hypoxic ischemic encephalopathy, severe, global. |
| 3 | Male | 58 | Hypoxic/ischemic injury, global, widespread |
| | | | astrogliosis/microgliosis. |
| 4 | Male | 84 | Dementia. Beta-amyloid plaques are noted in cortex and |
| | | | cerebellum. |
| 5 | Female | 80 | Severe hypoxic ischemic encephalopathy, severe. Global |
| | | | astrogliosis and microgliosis. Mild Alzheimer-type pathology. |
| 6 | Female | 74 | Acute hypoxic-ischemic encephalopathy, global, moderate to |
| | | | severe. Arteriolosclerosis, mild. Metabolic gliosis, moderate |
| 7 | Male | 66 | Left frontal subacute hemorrhagic infarct. Multifocal subacute |
| | | | infarcts in pons and left cerebral peduncle. Global astrogliosis |
| | | | and microgliosis (see microscopic description). Alzheimer's |
| | | | pathology. |
| 8 | Female | 76 | Hypoxic ischemic encephalopathy, moderate. Alzheimer's |
| | | | pathology. Atherosclerosis, moderate. Arteriolosclerosis, |
| | | | moderate |
| 9 | Male | 72 | Hypoxic/ischemic injury, acute to subacute, involving |
| | | | hippocampus, medulla and cerebellum. Mild atherosclerosis. |
| | | | Mild arteriolosclerosis |
| 10 | Male | 71 | Hypoxic-ischemic encephalopathy, acute, global, mild to |
| | | | moderate. Diffuse Lewy body disease, neocortical type, |
| | | | consistent with Parkinson disease dementia. Atherosclerosis, |
| | | | severe. Arteriolosclerosis, mild. |

### Lysate Preparation and Western Blots

Tissues (50 mg) were isotonically lysed using a Dounce homogenizer in 0.25 ml of 10 mM Tris Maleate (pH 7.0) buffer with protease inhibitors (Complete inhibitors from Roche). Samples were centrifuged at 8,000×g for 20 minutes and the protein concentrations of the supernatants were determined by Bradford assay. To determine protein levels in tissue lysates, tissue proteins (20 μg) were separated by 4-20% SDS-PAGE and immunoblots were developed using the following antibodies: pSMAD3 (Abcam, 1:1000), SMAD3 (Abcam, 1:1000), AMPK (Abcam, 1:1000), pAMPK (Abcam, 1:1000), CDK5 (Thermofisher, 1: 1,000), and p25 (Thermofisher, 1:1000), Tau (Thermofisher, 1:1000), pTauS199 (Thermofisher, 1:1000), pTauS202/T205 (Abcam, 1:1000), pTauS262 (Abcam, 1:1000), GSK3β Abcam, 1:1000), pGSK3βS9 (Abcam, 1:1000), pGSK3βT216 (Abcam, 1:1000), APP (Abcam, 1:1000), BACE1 (Abcam, 1:1000), GAPDH (Santa Cruz Biotech, 1:1000), CTF-β (Santa Cruz Biotech, 1:1000), Calbindin (Abcam, 1:1000), and GCPII (Thermofisher, 1:4000).

### Analyses of Ryanodine Receptor Complex

Tissue lysates (0.1 mg) were treated with buffer or 10 μM Rycal® Compound 1 at 4° C. RyR2 was immunoprecipitated from 0.1 mg lung, heart, and brain using an anti-RyR2 specific antibody (2 μg) in 0.5 ml of a modified radioimmune precipitation assay buffer (50 mm Tris-HCl, pH 7.2, 0.9% NaCl, 5.0 mm NaF, 1.0 mm Na₃VO₄, 1% Triton X-100, and protease inhibitors) (RIPA) overnight at 4° C. RyR2 specific antibody was an affinity-purified polyclonal rabbit antibody using the peptide CKPEFNNHKDYAQEK corresponding to amino acids 1367-1380 of mouse RyR2 with a cysteine residue added to the amino terminus. The immune complexes were incubated with protein A-Sepharose beads (Sigma) at 4° C. for 1 h, and the beads were washed three times with RIPA. The immunoprecipitates were size-fractionated on SDS-PAGE gels (4-20% for RyR2, calstabin2, and NOX2) and transferred onto nitrocellulose membranes for 1 h at 200 mA. Immunoblots were developed using the following primary antibodies: anti-RyR2 (Affinity Bioreagents, 1:2500), anti-phospho-RyR-Ser(pS)-2808 (Affinity Bioreagents 1:1000), anti-calstabin2 (FKBP12 C-19, Santa Cruz Biotechnology, Inc., 1:2500), and anti-NOX2 (Abcam, 1:1000). To determine channel oxidation, the carbonyl groups in the protein side chains were derivatized to DNP by reaction with 2,4-dinitrophenylhydrazine. The DNP signal associated with RyR2 was determined using a specific anti-DNP antibody according to the manufacturer's instructions (Millipore, Billerica, MA, 1:1000). All immunoblots were developed and quantified using an Odyssey system (LI-COR Biosciences, Lincoln, NE) with IR-labeled anti-mouse and anti-rabbit IgG (1:5000) secondary antibodies.

### Ryanodine Binding

RyR2 was immunoprecipitated from 1.5 mg of tissue lysate using an anti-RyR2 specific antibody (25 μg) in 1.0 ml of a modified RIPA buffer overnight at 4° C. The immune complexes were incubated with protein A-Sepharose beads (Sigma) at 4° C. for 1 h, and the beads were washed three times with RIPA buffer, followed by 2 washes with ryanodine binding buffer (10 mM Tris-HCl, pH 6.8, 1 M NaCl, 1% CHAPS, 5 mg/ml phosphatidylcholine, and protease inhibitors). Immunoprecipitates were incubated in 0.2 ml of binding buffer containing 20 nM [³H] ryanodine and either of 150 nM and 20 μm free Ca²⁺ for 1 h at 37° C. Samples were diluted with 1 ml of ice-cold washing buffer (25 mm Hepes, pH 7.1, 0.25 m KCl) and filtered through Whatman GF/B membrane filters pre-soaked with 1% polyethyleneimine in washing buffer. Filters were washed three times with 5 ml of washing buffer. The radioactivity remaining on the filters is determined by liquid scintillation counting to obtain bound [³H] ryanodine. Nonspecific binding was determined in the presence of 1000-fold excess of non-labeled ryanodine.

GSSH/GSH ratio measurement and SMAD3 phosphorylation

Approximately 20 mg of tissue suspended in 200 μL of ice-cold PBS/0.5% NP-40, pH6.0 was used for lysis. Tissue is homogenized with a Dounce homogenizer with 10-15 passes. Samples are centrifuged at 8,000×g for 15 minutes at 4° C. to remove any insoluble material. Supernatant is transferred to a clean tube. Deproteinizing of the samples is accomplished by adding 1 volume ice cold 100% (w/v) TCA into 5 volumes of sample and vortexing briefly to mix well. After incubating for 5 min on ice, samples are centrifuged at 12,000×g for 5 minutes at 4° C. and the supernatant is transferred to a fresh tube. The samples are neutralized by adding NaHCO₃ to the supernatant and vortexing briefly. Samples are centrifuged at 13,000×g for 15 minutes at 4° C. and supernatant is collected. Samples are now deproteinized, neutralized, TCA has been removed, and are ready to use in the assay. The GSSG/GSH is determined using the ratio detection assay kit (Abcam, ab138881). Briefly, in two separate assay reactions, GSH (reduced) is measured directly with a GSH standard and Total GSH (GSH+GSSG) is measured by using a GSSG standard. A 96-well plate is set up with 50 μL duplicate samples and standards with known concentrations of GSH and GSSG. A Thiol green indicator is added, and the plate is incubated for 60 min at room temperature. Fluorescence at Ex/Em=490/520 nm is measured with a fluorescence microplate reader and the GSSG/GSH for samples are determined comparing fluorescence signal of samples with known standards.

### Kynurenic Acid Assay

Kynurenic acid (KYNA) concentration in brain lysates was determined using an ELISA kit for KYNA (Immosol, Bordeaux, France). Briefly, samples (50 μl) are added to a microtiter plate designed to extract the KCNA from the samples. An Acylation Reagent was added for 90 min at 37° C. to derivatize the samples. After derivatization, 50 μl of the prepared standards and 100 μl samples were pipetted into the appropriate wells of the kynurenic acid microtiter plate. KYNA Antiserum was added to all wells and the plate was incubated overnight at 4° C. After washing the plate 4 times, the enzyme conjugate was added to each well. The plate was incubated for 30 min at RT on a shaker at 500 rpm. The enzyme substrate was added to all wells and the plate was incubated for 20 min at RT. Stop Solution was added to each well. A plate reader was used to determine the absorbance at 450 nm. The sample signals were compared to a standard curve.

### PKA Activity Assay

PKA activity in brain lysates was determined using a PKA activity kit (Thermofisher, EIAPKA). Briefly, samples were added to a microtiter plate containing an immobilized PKA substrate that is phosphorylated by PKA in the presence of ATP. After incubating the samples with ATP at room temperature for 2 h, the plate was incubated with the phospho-PKA substrate antibody for 60 min. After washing the plate with wash buffer, goat anti-rabbit IgG HRP conjugate was added to each well. The plate was aspirated, washed, and TMB substrate was added to each well, which was then incubated for 30 min at room temperature. A plate reader was used to determine the absorbance at 450 nm. The sample signals were compared to a standard curve.

### CAMKII Activity Assay

CAMKII activity in brain lysates was determined using the CycLex CaM kinase II Assay Kit (MBL international). Briefly, samples were added to a microtiter plate containing an immobilized CAMKII substrate that is phosphorylated by CAMKII in the presence of Mg²⁺ and ATP. After incubating the samples in kinase buffer containing Mg²⁺ and ATP at room temperature for 1 h, the plate was washed and incubated with the HRP conjugated anti-phospho-CAMKII substrate antibody for 60 min. The plate was aspirated, washed, and TMB substrate was added to each well, which was then incubated for 30 min at room temperature. A plate reader was used to determine the absorbance at 450 nm. The sample signals were compared to a standard curve.

### Statistics

Group data are presented as mean±SD. Statistical comparisons between the two groups were determined using an unpaired t-test. Values of p<0.05 were considered statistically significant. All statistical analyses were performed with Prism 8.0.

### Results

### Oxidative Stress and TGF-β, PKA, and CAMKII Activation

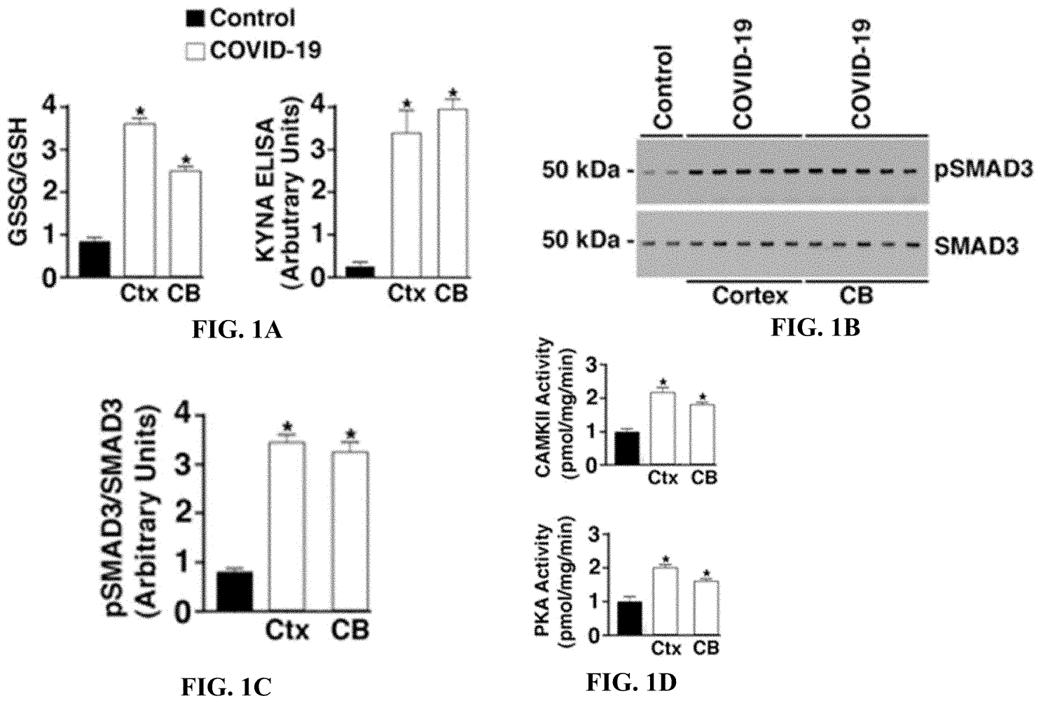

Oxidative stress levels were determined in brain tissues (cortex, cerebellum) from COVID-19 patient autopsy tissues and controls by measuring the ratio of glutathione disulfide (GSSG) to glutathione (GSH) by an ELISA kit. COVID-19 patients exhibited significant oxidative stress with a 3.8- and 3.2-fold increase in GSSG/GSH ratios in cortex (Ctx) and cerebellum (CB) compared to controls, respectively (FIG. 1A). Kynurenic acid levels in the Ctx and CB were measured using an ELISA kit. COVID-19 brains had a significant increase in the Ctx and CB compared to controls (FIG. 1A). An additional marker of tissue inflammation is increased cytokine expression. SMAD3 phosphorylation, a downstream signal of TGFβ, was increased in COVID-19 Ctx and CB tissue lysates compared to controls (FIGS. 1B and 1C). Increased adrenergic activation in the brain by patients infected with SARS-CoV-2 was also demonstrated by measuring PKA activity in the Ctx and CB and CaMKII activity was increased as well (FIG. 1D).

### Activation of Alzheimer's Disease-Linked Signaling

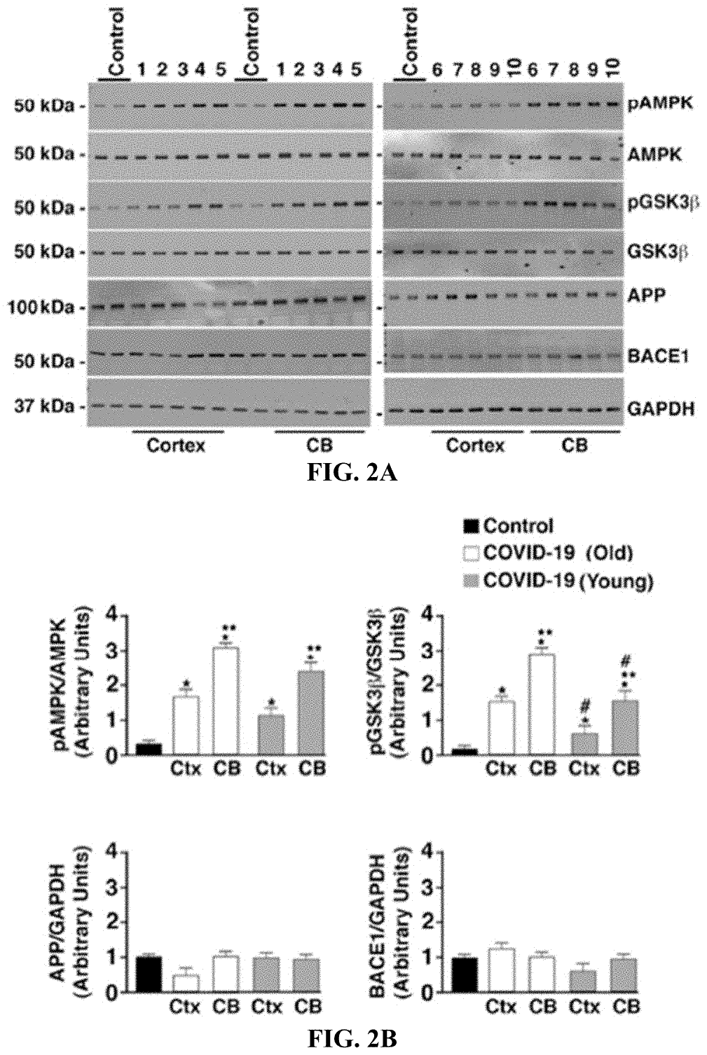

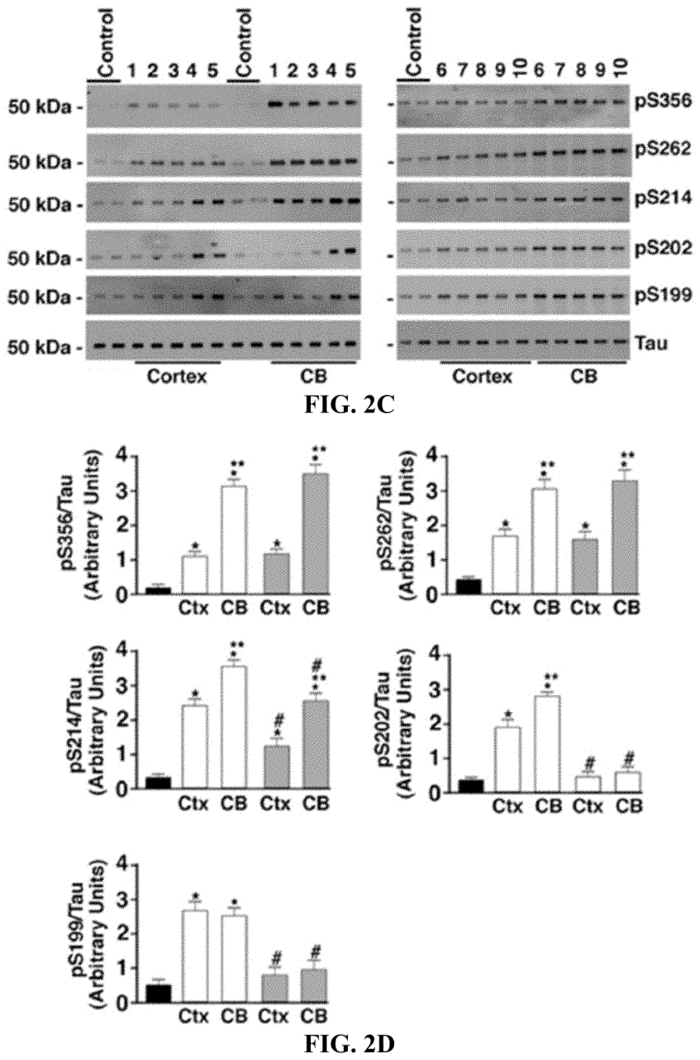

Both PKA and CaMKII have been directly implicated in the increased phosphorylation of Tau associated with Alzheimer's Disease. Since COVID-19 brain lysates had increased PKA and CaMKII activity, Alzheimer's Disease-linked biochemistry was evaluated in the COVID-19 brain lysates. Normal amyloid precursor protein processing (APP) was observed in COVID-19 brain lysates as demonstrated by normal BACE1 and APP levels compared to controls (FIGS. 2A and 2B). Abnormal APP processing was only observed in lysates from patients with diagnosed Alzheimer's Disease (see Table 1 for patient details). However, phosphorylation/activation of AMPK and GSK3β was observed in SARS-CoV-2 infected patient brain lysates. Activation of these kinases along with the activation of PKA and CAMKII (FIG. 1) leads to a hyperphosphorylation of Tau at multiple residues (FIGS. 2C and D) even though Tau hyperphosphorylation in the cerebellum is not typical of Alzheimer's Disease pathology.

### RyR2 Channel Oxidation and Leak

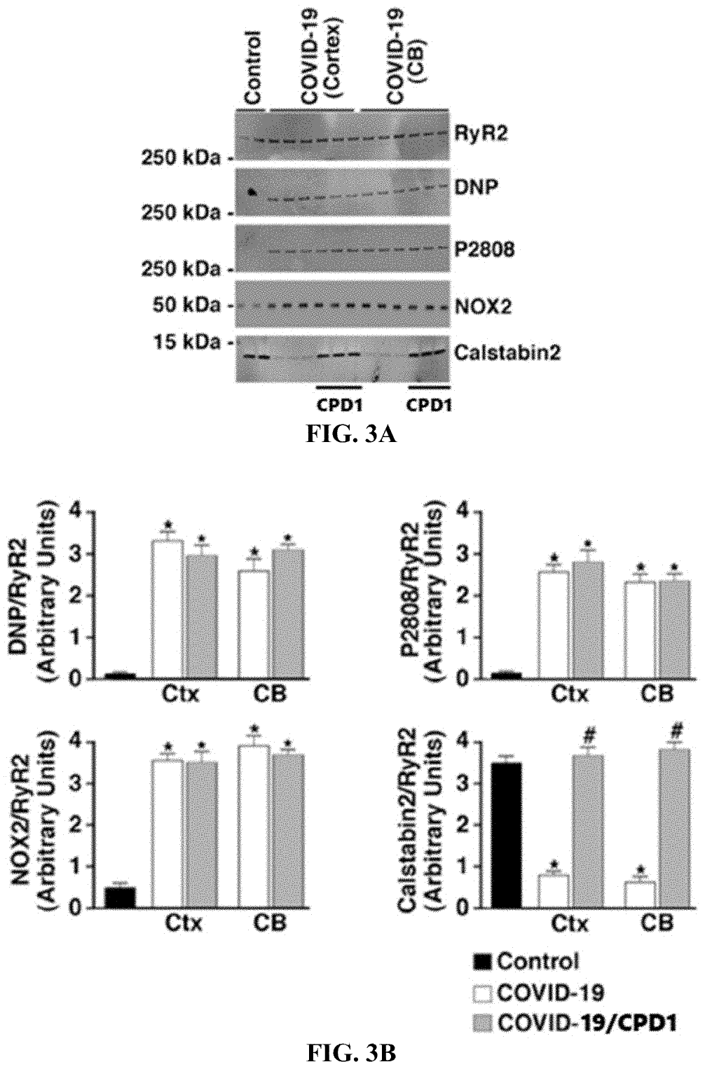

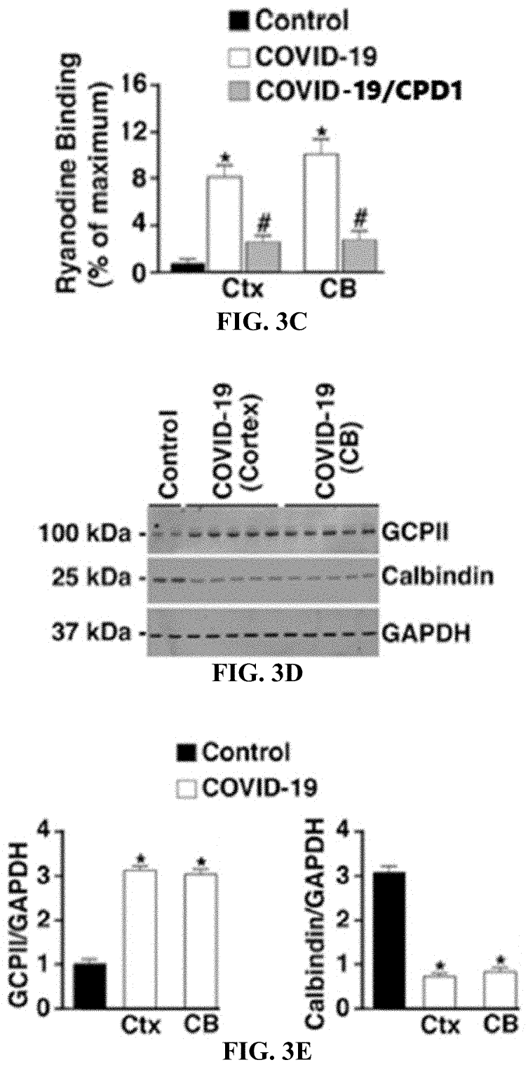

RyR2 biochemistry was investigated to determine if RyR2 in COVID-19 brain tissues are “leaky”. Increased NOX2/RyR2 binding was shown in Ctx and CB lysates from SARS-CoV-2 infected individuals compared to controls using co-immunoprecipitation (FIGS. 3A and B). In addition, RyR2 from SARS-CoV-2 infected brains had increased oxidation, increased Serine 2808 PKA phosphorylation, and depletion of the stabilizing protein subunit calstabin2 compared to controls (FIGS. 3A and B). RyR channels exhibiting these characteristics can be inappropriately activated at low cytosolic Ca²⁺ concentrations resulting in a pathological ER/SR Ca²⁺ leak. ³[H]Ryanodine binding to immunoprecipitated RyR2 was measured at both 150 nM and 20 μM free Ca²⁺. Since ryanodine binds only to the open state of the channel under these conditions, ³[H]Ryanodine binding may be used as a surrogate measure of channel open probability. The total amount of RyR immunoprecipitated was the same for control and COVID-19 samples (data not shown). Increased RyR2 channel activity at resting conditions (150 nM free Ca²⁺) was observed in COVID-19 channels compared to controls (FIG. 3C). Under these conditions, RyR channels should be closed. Rebinding of calstabin2 to RyR2, using a Rycal® compound, has been shown to reduce SR/ER Ca²⁺ leak, despite the persistence of the channel remodeling. Indeed, calstabin2 binding to RyR2 was increased when COVID-19 patient brain tissue lysates were treated ex-vivo with the Rycal® Compound 1 (“CPD1”) (FIGS. 3A and B). Abnormal RyR2 activity observed at resting Ca²⁺ concentration was also decreased by treatment with the Rycal® compound (FIG. 3C).

A finding concerning the Tau phosphorylation in brain lysates from SARS-CoV-2 patients was the increased of phosphorylation at multiple sites in the cerebellum. This is atypical of Alzheimer's Disease. One potential mechanism to explain this finding is the significantly decreased levels of calbindin expressed in COVID-19 cerebellum (FIG. 3D). The decreased cerebellar calbindin levels could make this area of the brain more susceptible to Ca²⁺-induced activation of enzymes upstream of Tau phosphorylation. Moreover, increased GCPII expression was observed in COVID-19 cortex and cerebellar lysates (FIG. 3D), which would reduce mGluR3 inhibition of PKA signaling and could contribute to the PKA hyperphosphorylation of RyR2.

### Model for the Role for Leaky RyR2 in the Pathophysiology of SARS-CoV-2 Infection

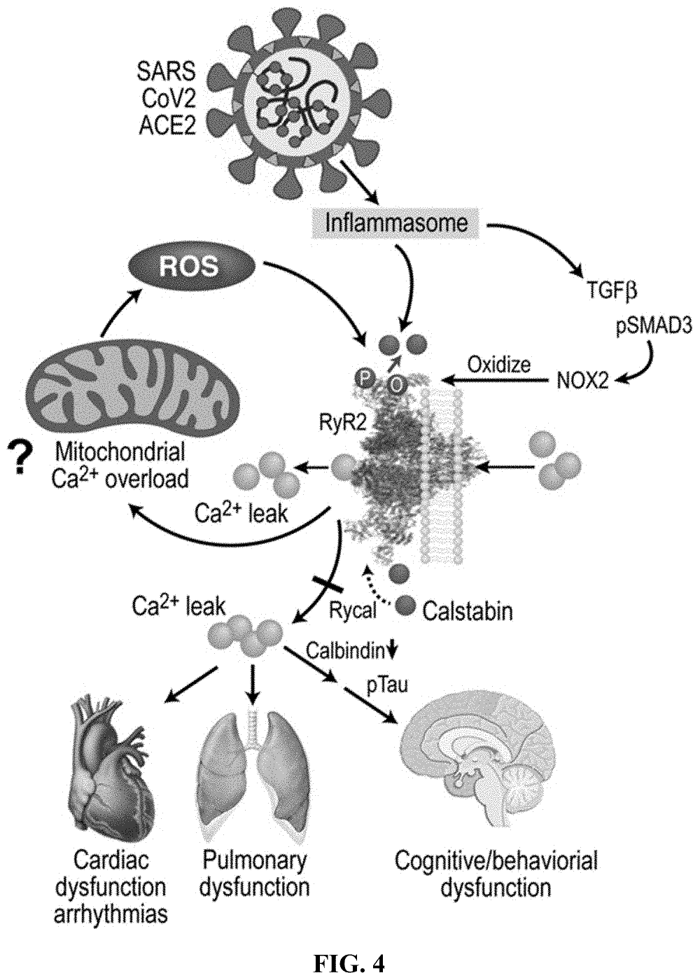

A role was found for leaky RyR2 in the pathophysiology of SARS-CoV-2 infection. As shown in FIG. 4, SARS-CoV-2 infection targets cells via the ACE2 receptor, inducing inflammasome stress response/activation of stress signaling pathways. This results in increased TGF-β signaling, which activates SMAD3 (pSMAD) and increases NOX2 expression and the amount of NOX2 associated with RyR2. Increased NOX2 activity at RyR2 oxidizes the channel, causing calstabin2 depletion from the channel macromolecular complex, destabilization of the closed state, and ER/SR calcium leak contributes to cardiac dysfunction, arrhythmias, pulmonary insufficiency, and cognitive and behavioral abnormalities associated with neurodegenreation. Decreased calbindin in COVID-19 is anticipated to render brain more susceptible to Tau pathology. Rycal® compounds fix the RyR2 channel leak by restoring calstabin2 binding and stabilizing the channel closed state. Thus, fixing leaky RyR2 is expected to improve cardiac, pulmonary, and cognitive function in COVID-19.

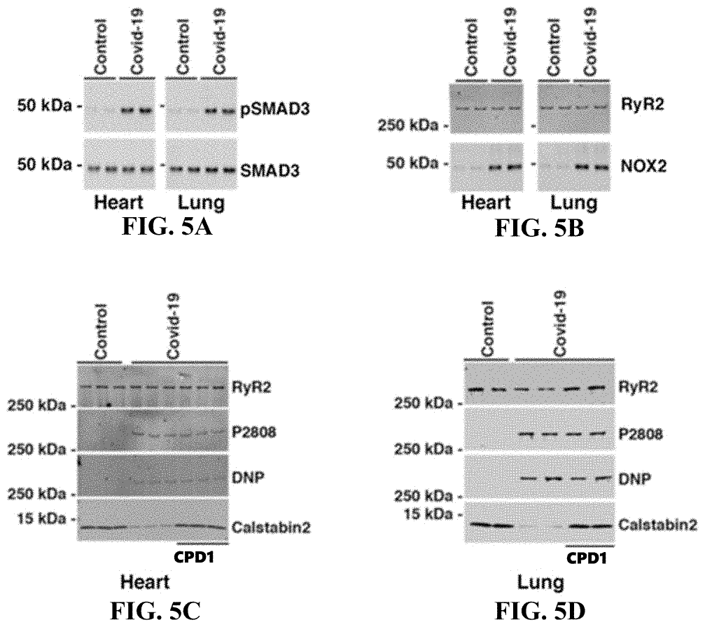

In addition to the brain of COVID-19 patients, increased systemic oxidative stress and activation of the TGF-β signaling pathway were manifested in lung, and heart, and that correlates with oxidation-driven biochemical remodeling of RyR2 (FIGS. 3 and 5). The effect of Rycal® Compound 1 (“CPD1”) on lung and heart tissue of COVID-19 patients is shown in the Western blots depicting RyR2 oxidation, PKA phosphorylation, and calstabin2 bound to the channel in FIG. 5C (heart) and 5D (lung). This RyR2 remodeling results in intracellular Ca2+ leak which can play a role in heart failure progression, pulmonary insufficiency, as well as cognitive dysfunction. The alteration of cellular Ca²⁺ dynamics has also been implicated in COVID-19 pathology. Taken together, the present data suggest that leaky RyR2 may play a role in the long-term sequelae of COVID-19, including the “brain fog” associated with SARS-CoV-2 infection which also could be a forme fruste of Alzheimer's Disease, and could predispose long COVID patients to developing Alzheimer's Disease later in life. Leaky RyR2 channels may be a therapeutic target for amelioration of some of the persistent cognitive deficits associated with long COVID-19.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

We claim:

1. A method of treating cognitive dysfunction associated with a respiratory virus infection, the method comprising administering a therapeutically effective amount of a calcium channel stabilizer to a subject in need thereof, the calcium channel stabilizer comprising a 1,4-benzothiazepine moiety.

2. The method of claim 1, wherein the respiratory virus is a coronavirus.

3. The method of claim 1, wherein the respiratory virus is selected from the group consisting of severe acute respiratory syndrome (SARS) coronavirus (SARS-CoV), SARS-CoV-2 (COVID-19), Middle East Respiratory Syndrome (MERS), respiratory syncytial virus (RSV), influenza virus, parainfluenza virus (NV), pneumovirus (PMV), metapneumovirus (MPV), respirovirus, and rubulavirus.

4. The method of claim 1, wherein the respiratory virus is SARS-CoV-19.

5. The method of claim 1, wherein the treating decreases calcium leak from a RyR2 channel of the subject.

6. The method of claim 1, wherein the treating increases RyR2-Castabin2 binding in cardiac muscle of the subject.

7. The method of claim 1, wherein the treating decreases open probability (Pₒ) of RyR2 protein in the subject.

8. The method of claim 1, wherein the cognitive dysfunction comprises a deficit in attention, executive functioning, language, processing speed, memory, and any combination thereof.

9. The method of claim 1, wherein the calcium channel stabilizer comprises the following structural formula 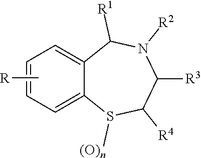
wherein,
n is 0, 1, or 2;
R is located at one or more positions on the benzene ring; each R is independently selected from the group consisting of H, halogen, —OH, —NH₂, —NO₂, —CN, —N₃, —SO₃H, acyl, alkyl, alkoxyl, alkylamino, cycloalkyl, heterocyclyl, heterocyclylalkyl, alkenyl, (hetero-)aryl, (hetero-)arylthio, and (hetero-)arylamino; wherein each acyl, alkyl, alkoxyl, alkylamino, cycloalkyl, heterocyclyl, heterocyclylalkyl, alkenyl, (hetero-)aryl, (hetero-)arylthio, and (hetero-)arylamino may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
R¹ is selected from the group consisting of H, oxo, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
R² is selected from the group consisting of —C═O(R⁵), —C═S(R⁶), —SO₂R⁷, —POR⁸R⁹, —(CH₂)ₘ—R¹⁰, alkyl, aryl, heteroaryl, cycloalkyl, cycloalkylalkyl, and heterocyclyl; wherein each alkyl, aryl, heteroaryl, cycloalkyl, cycloalkylalkyl, and heterocyclyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
R³ is selected from the group consisting of H, —CO₂Y, —CONY, acyl, alkyl, alkenyl, aryl, cycloalkyl, and heterocyclyl; wherein each acyl, alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl; and wherein Y is selected from the group consisting of H, alkyl, aryl, cycloalkyl, and heterocyclyl;
R⁴ is selected from the group consisting of H, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
R⁵ is selected from the group consisting of —NR¹⁶, NHNHR¹⁶, NHOH, —OR¹⁵, —CONH₂NHR¹⁶, —CO₂R¹⁵, CONR¹⁶, —CH₂X, acyl, alkenyl, aryl, cycloalkyl, cycloalkylalkyl, heterocyclyl, and heterocyclylalkyl; wherein each acyl, alkenyl, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
R⁶ is selected from the group consisting of —OR¹⁵, —NHNR¹⁶, —NHOH, —NR¹⁶, —CH₂X, acyl, alkenyl, alkyl, aryl, cycloalkyl, cycloalkylalkyl, heterocyclyl, and heterocyclylalkyl; wherein each acyl, alkenyl, alkyl, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
R⁷ is selected from the group consisting of —OR¹⁵, —NR¹⁶, —NHNHR¹⁶, —NHOH, —CH₂X, alkyl, aryl, cycloalkyl, cycloalkylalkyl, heterocyclyl, and heterocyclylalkyl; wherein each alkyl, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
R⁸ and R⁹ independently are selected from the group consisting of OH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
R¹⁰ is selected from the group consisting of NH₂, —OH, —SO₂R¹¹, —NHSO₂R¹¹, —C═O(R₁₂), —NHC═O(R¹²), —OC═O(R¹²), and —POR¹³R¹⁴;
R¹¹, R¹², R¹³, and R¹⁴ independently are selected from the group consisting of H, —OH, —NH₂, —NHNH₂, —NHOH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
X is selected from the group consisting of halogen, —CN, —CO₂R¹⁵, —CONR¹⁶, —NR¹⁶, —OR¹⁵, —SO₂R⁷, and —POR⁸R⁹; and
R¹⁵ and R¹⁶ independently are selected from the group consisting of H, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
or a pharmaceutically-acceptable salt, hydrate, solvate, complex, or prodrug thereof.

10. The method of claim 1, wherein the calcium channel stabilizer comprises the following structural formula 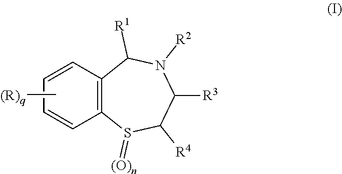
wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)NR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
n is 0, 1, or 2;
q is 0, 1, 2, 3, or 4;
t is 1, 2, 3, 4, 5, or 6; and
m is 1, 2, 3, or 4,
or a pharmaceutically-acceptable salt, hydrate, solvate, complex, or prodrug thereof.

11. The method of claim 10, wherein the calcium channel stabilizer is selected from the group consisting of: 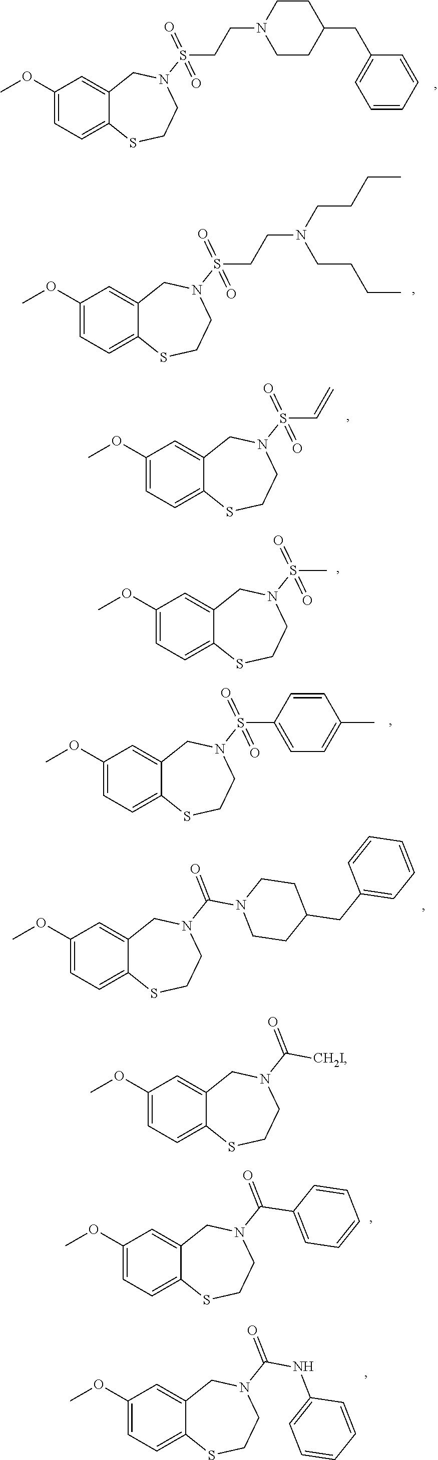 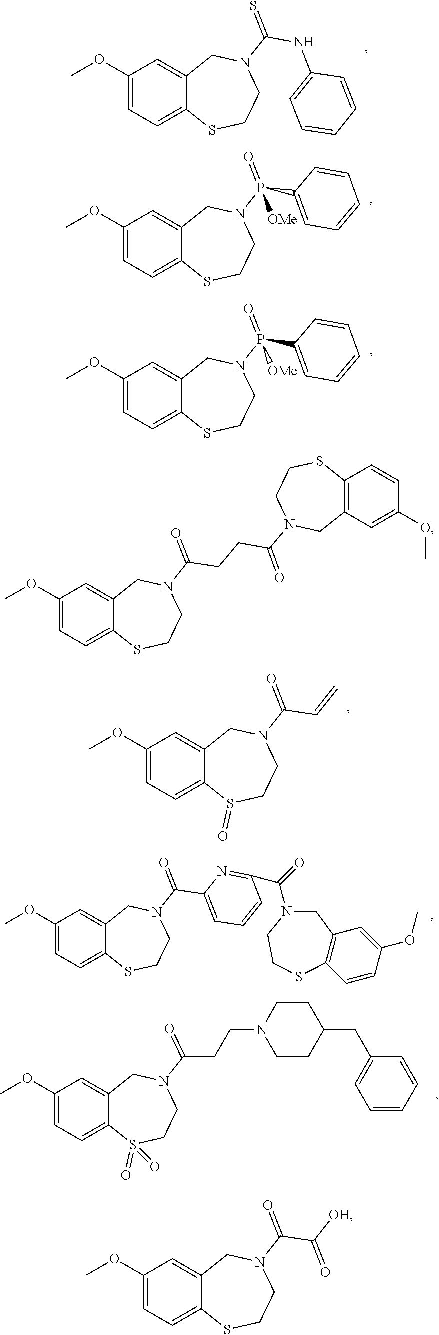 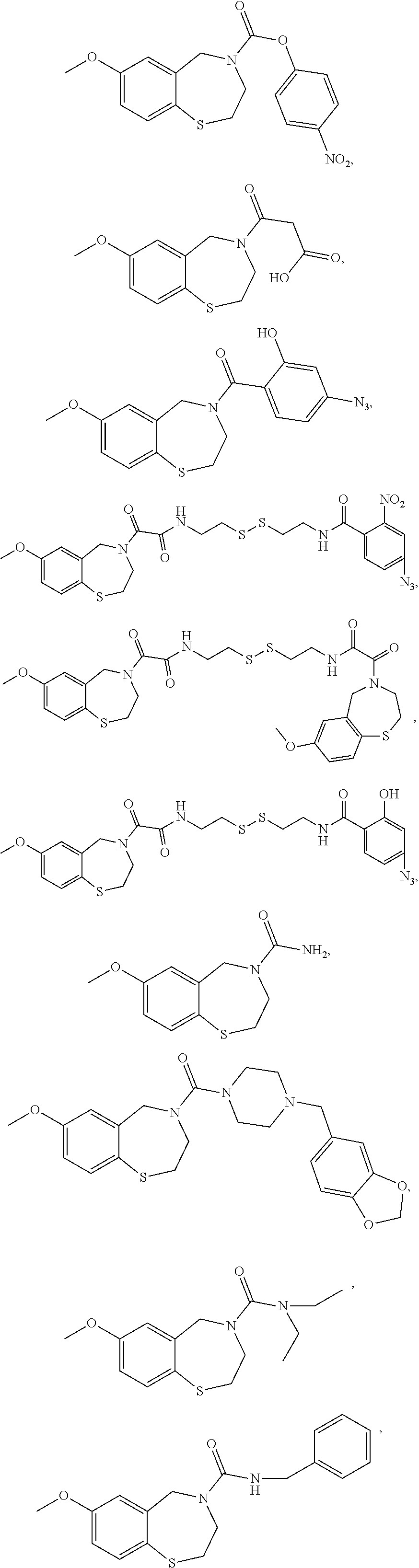 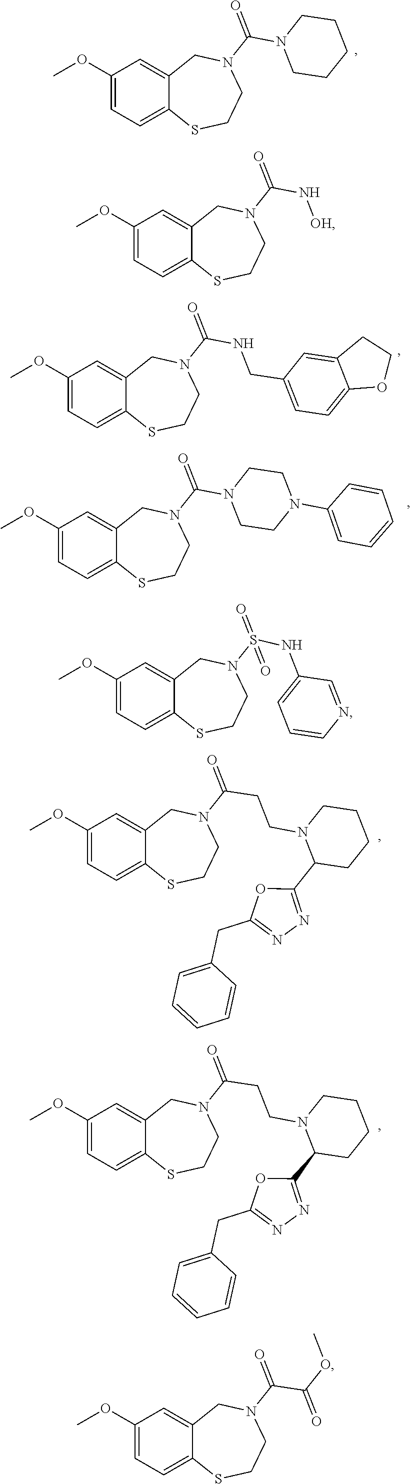 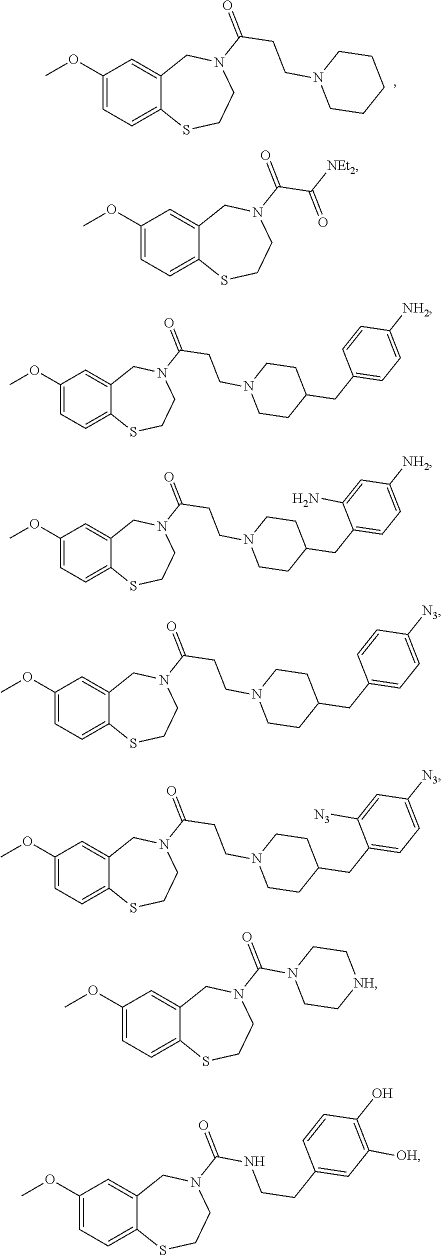 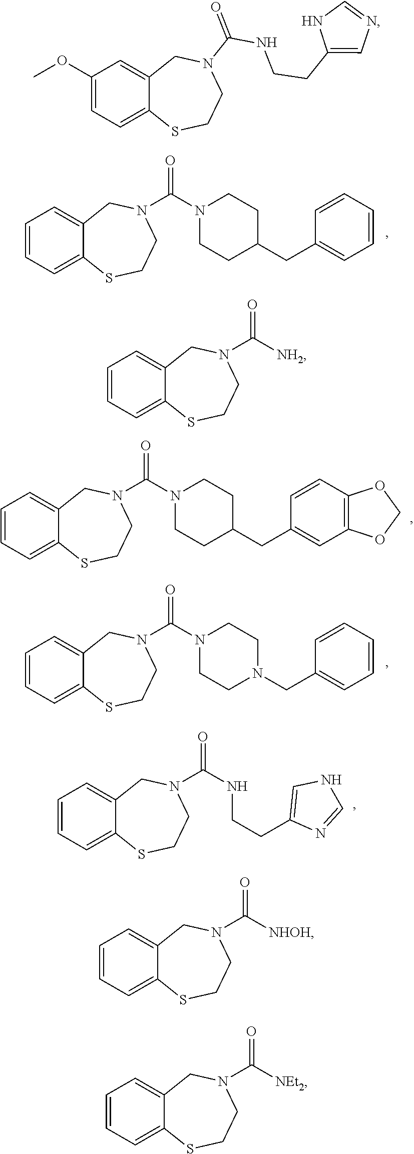 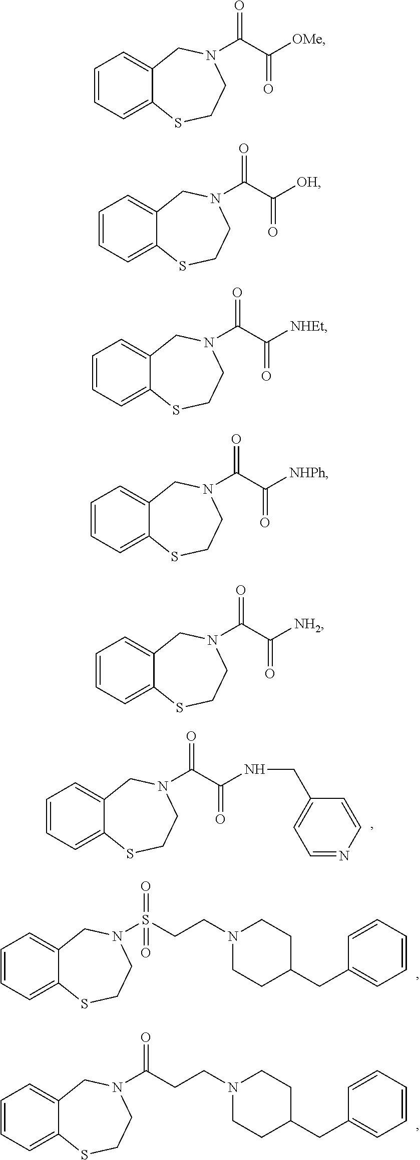 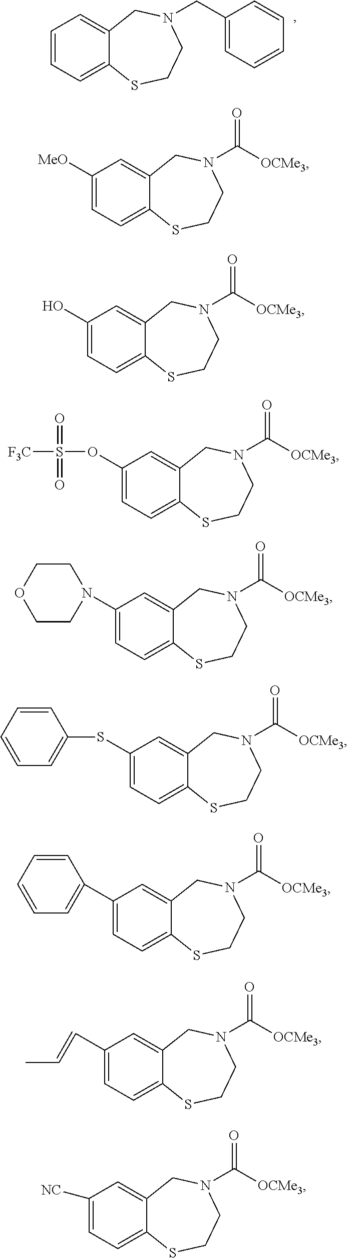 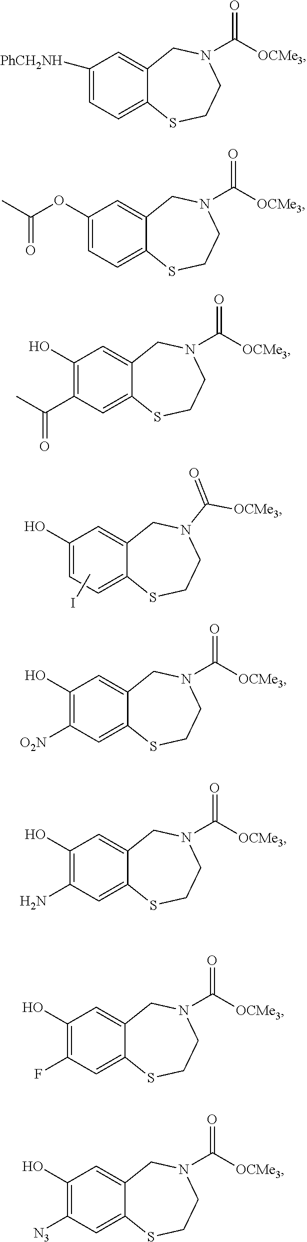 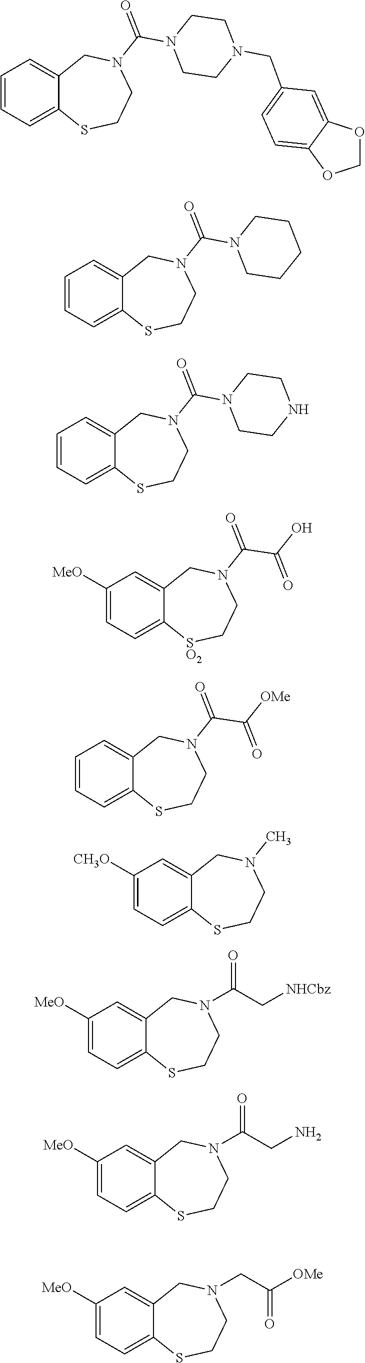 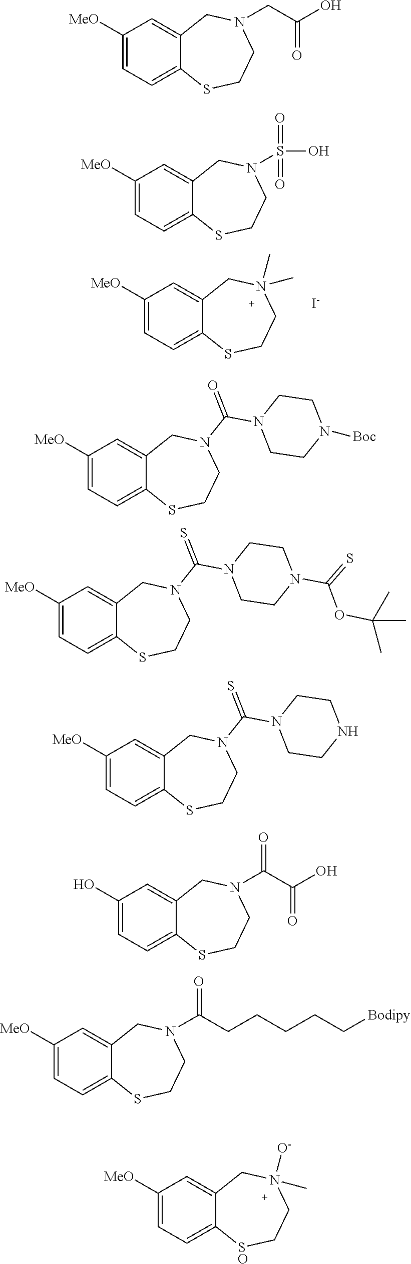 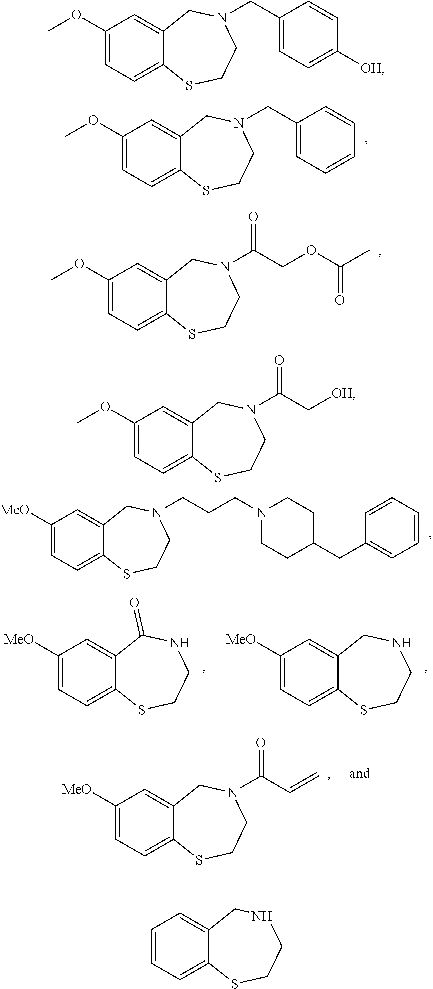
or a pharmaceutically-salt, hydrate, solvate, complex, or prodrug thereof.

12. The method of claim 1, wherein the calcium channel stabilizer is: 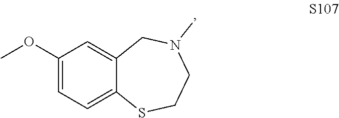
or a pharmaceutically-acceptable salt thereof.

13. The method of claim 1, wherein the calcium channel stabilizer comprises the following structural formula 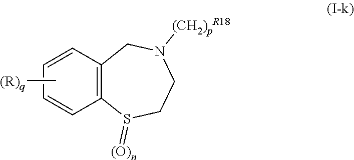
wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹⁸ is alkyl, aryl, cycloalkyl, or heterocyclyl, each of which is independently substituted or unsubstituted; or —NR¹⁵R¹⁶, —C(═O)NR¹⁵R¹⁶, —(C═O)OR¹⁵, or —OR¹⁵;
q is 0, 1, 2, 3, or 4;
p is 1, 2, 3, 4, 5, 6, 7, 8 9, or 10; and
n is 0, 1, or 2,
or a pharmaceutically-acceptable salt thereof.

14. The method of claim 1, wherein the calcium channel stabilizer comprises the following structural formula 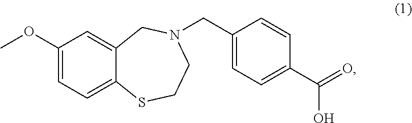 [SMILES: COc1ccc2c(c1)CN(Cc1ccc(C(=O)O)cc1)CCS2]
or a pharmaceutically-acceptable salt thereof.

15. The method of claim 1, wherein the calcium channel stabilizer is orally administered to the subject.

16. The method of claim 1, wherein the calcium channel stabilizer is administered in a pharmaceutical composition, the pharmaceutical composition further comprising at least one pharmaceutically acceptable excipient.

17. A method of treating cognitive dysfunction associated with a respiratory virus infection, the method comprising administering a therapeutically effective amount of a calcium channel stabilizer to a subject in need thereof, the calcium channel stabilizer represented by the structure: 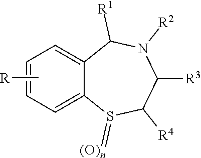
wherein,
n is 0, 1, or 2;
R is located at one or more positions on the benzene ring; each R is independently selected from the group consisting of H, halogen, —OH, —NH₂, —NO₂, —CN, —N₃, —SO₃H, acyl, alkyl, alkoxyl, alkylamino, cycloalkyl, heterocyclyl, heterocyclylalkyl, alkenyl, (hetero-)aryl, (hetero-)arylthio, and (hetero-)arylamino; wherein each acyl, alkyl, alkoxyl, alkylamino, cycloalkyl, heterocyclyl, heterocyclylalkyl, alkenyl, (hetero-)aryl, (hetero-)arylthio, and (hetero-)arylamino may be substituted with one or more radicals independently selected from the group consisting of halogen, —N, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
R¹ is selected from the group consisting of H, oxo, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
R² is selected from the group consisting of —C═O(R⁵), —C═S(R⁶), —SO₂R⁷, —POR⁸R⁹, —(CH₂)ₘ—R¹⁰, alkyl, aryl, heteroaryl, cycloalkyl, cycloalkylalkyl, and heterocyclyl; wherein each alkyl, aryl, heteroaryl, cycloalkyl, cycloalkylalkyl, and heterocyclyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
R³ is selected from the group consisting of H, —CO₂Y, —CONY, acyl, alkyl, alkenyl, aryl, cycloalkyl, and heterocyclyl; wherein each acyl, alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl; and wherein Y is selected from the group consisting of H, alkyl, aryl, cycloalkyl, and heterocyclyl;
R⁴ is selected from the group consisting of H, alkyl, alkenyl, aryl, cycloalkyl, and heterocyclyl; wherein each alkyl, alkenyl, aryl, cycloalkyl, and heterocyclyl may be substituted with one or more radicals independently selected from the group consisting of halogen, —N—, —O—, —S—, —CN, —N₃, —SH, nitro, oxo, acyl, alkyl, alkoxyl, alkylamino, alkenyl, aryl, (hetero-)cycloalkyl, and (hetero-)cyclyl;
R⁵ is selected from the group consisting of —NR¹⁶, NHNHR¹⁶, NHOH, —OR¹⁵, —CONH₂NHR¹⁶, —CO₂R¹⁵, CONR¹⁶, —CH₂X, acyl, alkenyl, aryl, cycloalkyl, cycloalkylalkyl, heterocyclyl, and heterocyclylalkyl; wherein each acyl, alkenyl, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
R⁶ is selected from the group consisting of —OR¹⁵, —NHNR¹⁶, —NHOH, —NR¹⁶, —CH₂X, acyl, alkenyl, alkyl, aryl, cycloalkyl, cycloalkylalkyl, heterocyclyl, and heterocyclylalkyl; wherein each acyl, alkenyl, alkyl, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
R⁷ is selected from the group consisting of —OR¹⁵, —NR¹⁶, —NHNHR¹⁶, —NHOH, —CH₂X, alkyl, aryl, cycloalkyl, cycloalkylalkyl, heterocyclyl, and heterocyclylalkyl; wherein each alkyl, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
R⁸ and R⁹ independently are selected from the group consisting of OH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkyl, alkoxyl, alkylamino, alkenyl, aryl, (hetero-)cycloalkyl, and (hetero-)cyclyl;
R¹⁰ is selected from the group consisting of NH₂, —OH, —SO₂R¹¹, —NHSO₂R¹¹, —C═O(R₁₂), —NHC═O(R¹²), —OC═O(R¹²), and —POR¹³R¹⁴;
R¹¹, R¹², R¹³, and R¹⁴ independently are selected from the group consisting of H, —OH, —NH₂, —NHNH₂, —NHOH, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
X is selected from the group consisting of halogen, —CN, —CO₂R¹⁵, —CONR₁₆, —NR₁₆, —OR¹⁵, —SO₂R⁷, and —POR⁸R⁹; and
R¹⁵ and R¹⁶ independently are selected from the group consisting of H, acyl, alkenyl, alkoxyl, alkyl, alkylamino, aryl, cycloalkyl, cycloalkylalkyl, heterocyclyl, and heterocyclylalkyl; wherein each acyl, alkenyl, alkoxyl, alkyl, alkylamino, aryl, cycloalkyl, cycloalkylalkyl, heterocyclyl, and heterocyclylalkyl may be substituted with one or more radicals independently selected from the group consisting of halogen, —N—, —O—, —S—, —CN, —N₃, nitro, oxo, acyl, alkenyl, alkoxyl, alkyl, alkylamino, amino, aryl, cycloalkyl, cycloalkylalkyl, heterocyclyl, heterocyclylalkyl, and hydroxyl;
or a pharmaceutically-acceptable salt, hydrate, solvate, complex, or prodrug thereof.

18. A method of treating cognitive dysfunction associated with a respiratory virus infection, the method comprising administering a therapeutically effective amount of a calcium channel stabilizer to a subject in need thereof, the calcium channel stabilizer represented by the structure: 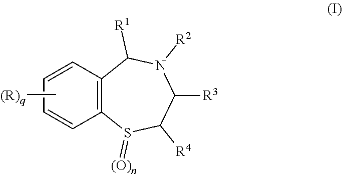
wherein:
each R is independently acyl, —O-acyl, alkyl, alkoxyl, alkylamino, alkylarylamino, alkylthio, cycloalkyl, alkylaryl, aryl, heteroaryl, heterocyclyl, heterocyclylalkyl, alkenyl, alkynyl, arylthio, arylamino, heteroarylthio, or heteroarylamino, each of which is independently substituted or unsubstituted; or halogen, —OH, —NH₂, —NO₂, —CN, —CF₃, —OCF₃, —N₃, —SO₃H, —S(═O)₂alkyl, —S(═O)alkyl, or —OS(═O)₂CF₃;
R¹ is alkyl, alkenyl, aryl, alkylaryl, cycloalkyl, heteroaryl, or heterocyclyl, each of which is independently substituted or unsubstituted; or H;
R² is alkyl, aryl, alkylaryl, heteroaryl, cycloalkyl, cycloalkylalkyl, or heterocyclyl, each of which is independently substituted or unsubstituted; or H, —C(═O)R⁵, —C(═S)R⁶, —SO₂R⁷, —P(═O)R⁸R⁹, or —(CH₂)ₘ—R¹⁰;
R³ is acyl, —O-acyl, alkyl, alkenyl, aryl, alkylaryl, cycloalkyl, heteroaryl, or heterocyclyl, each of which is independently substituted or substituted; or H, —CO₂Y, or —C(═O)NHY;
Y is alkyl, aryl, alkylaryl, cycloalkyl, heteroaryl, or heterocyclyl, each of which is independently substituted or unsubstituted; or H;
R⁴ is alkyl, alkenyl, aryl, alkylaryl, cycloalkyl, heteroaryl, or heterocyclyl, each of which is independently substituted or unsubstituted; or H;
each R⁵ is acyl, alkyl, alkenyl, aryl, alkylaryl, cycloalkyl, cycloalkylalkyl, heteroaryl, heterocyclyl, or heterocyclylalkyl, each of which is independently substituted or unsubstituted; or —NR¹⁵R¹⁶, —(CH₂)ₜNR¹⁵R¹⁶, —NHNR¹⁵R¹⁶, —NHOH, —OR¹⁵, —C(═O)NHNR¹⁵R¹⁶, —CO₂R¹⁵, —C(═O)NR¹⁵R¹⁶, or —CH₂X;
each R⁶ is acyl, alkenyl, alkyl, aryl, alkylaryl, cycloalkyl, cycloalkylalkyl, heteroaryl, heterocyclyl, or heterocyclylalkyl, each of which is independently substituted or unsubstituted; or —OR¹⁵, —NHNR¹⁵R¹⁶, —NHOH, —NR¹⁵R¹⁶, or —CH₂X;
each R⁷ is alkyl, alkenyl, alkynyl, aryl, alkylaryl, cycloalkyl, cycloalkylalkyl, heteroaryl, heterocyclyl, or heterocyclylalkyl, each of which is independently substituted or unsubstituted; or —OR¹⁵, —NR¹⁵R¹⁶, —NHNR¹⁵R¹⁶, —NHOH, or —CH₂X;
each R⁸ and R⁹ are each independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or OH;
each R¹⁰ is —NR¹⁵R¹⁶, OH, —SO₂R¹¹, —NHSO₂R¹¹, C(═O)(R¹²), NHC═O(R¹²), —OC═O(R¹²), or —P(═O)R¹³R¹⁴;
each R¹¹, R¹², R¹³, and R¹⁴ is independently acyl, alkenyl, alkoxyl, alkyl, alkylamino, aryl, alkylaryl, cycloalkyl, cycloalkylalkyl, heteroaryl, heterocyclyl, or heterocyclylalkyl, each of which is independently substituted or unsubstituted; or H, OH, NH₂, —NHNH₂, or —NHOH;
each X is independently halogen, —CN, —CO₂R¹⁵, —C(═O)NR¹⁵R¹⁶, —NR¹⁵R¹⁶, —OR¹⁵, —SO₂R⁷, or —P(═O)R⁸R⁹;
each R¹⁵ and R¹⁶ is independently acyl, alkenyl, alkoxyl, OH, NH₂, alkyl, alkylamino, aryl, alkylaryl, cycloalkyl, cycloalkylalkyl, heteroaryl, heterocyclyl, or heterocyclylalkyl, each of which is independently substituted or unsubstituted, or H; or R¹⁵ and R¹⁶ together with the N to which R¹⁵ and R¹⁶ are bonded form a heterocycle that is substituted or unsubstituted;
n is 0, 1, or 2;
q is 0, 1, 2, 3, or 4;
t is 1, 2, 3, 4, 5, or 6; and
m is 1, 2, 3, or 4,
or a pharmaceutically-acceptable salt, hydrate, solvate, complex, or prodrug thereof.
